(19) United States
(12) Patent Application Publication
(10) Pub. No.: US 2005/0107775 A1
(43) Pub. Date: May 19, 2005

(54) Method and apparatus for controlling ablation in refractive surgery

(75) Inventors: David Huang, Cleveland Heights, OH (US); Raj Shekhar, Sagamore Hills, OH (US); Maolong Tang, Columbus, OH (US)

Correspondence Address: Richard S. Wesorick;Tarolli Sundheim Covell & Tummino, 526 Superior Avenue, Suite 1111, Cleveland, OH, 44114, US

(73) Assignee: The Cleveland Clinic Foundation, Cleveland, OH (US)

(21) Appl. No.: 10/506,842

(22) PCT Filed: Feb. 28, 2003
(86) PCT No.: PCT/US03/06343

Related U.S. Application Data
(60) Provisional application No. 60/361,474, filed on Mar. 4, 2002.

(51) Int. Cl. A61B 18/20

(52) U.S. Cl. 606/5; 606/11

(57) ABSTRACT

The present invention relates to laser ablation patterns to correct refractive errors of the eye (60) such as nearsightedness, farsightedness, astigmatism, and higher order aberrations of the eye (60). The laser ablation patterns used to control the laser (10) prevent induced aberrations by compensating for post-procedure epithelial smoothing. The position of laser pulses (12) is also controlled to optimize the achievement of the intended ablation pattern.

### BACKGROUND OF THE INVENTION

Laser refractive surgery is often used to correct refractive errors, such as myopia, hyperopia, and astigmatism, in a patient's eyes. Refractive errors are also called lower-order aberrations of the eye. The eye also may have higher-order aberrations caused by irregularities in the cornea or crystalline lens. More recently, laser correction is also applied to higher-order aberrations such as coma, spherical aberrations, and other aberrations. Ideally, the laser refractive surgery should accurately remove the refractive error and not induce additional aberrations. The refractive errors and aberrations are removed through corneal ablation. The laser device follows a predetermined ablation pattern designed to correct the refractive errors and aberrations. These patterns are referred to as ablation patterns.

If the laser ablation patterns used in photorefractive keratectomy (PRK) and laser in-situ keratomileusis (LASIK) procedures to correct the refractive error are not properly selected, post-procedure aberrations can result that reduce quality of vision in scotopic conditions. These resulting post-procedure aberrations are not inherent in the predetermined ablation patterns used to correct the refractive error, but are believed to result from the corneal healing response after surgery. Such post-procedure or post-operative aberrations are referred to herein as “induced aberrations.”

Known laser ablation algorithms for vision correction have assumed an ideal target spherical surface. This is known in the art to be the Munnerlyn approach. The pre-operative corneal shape is approximated by the closest spherocylindrical shape. The ablation pattern is the difference between the assumed ideal target shape and the pre-operative spherocylindrical shape. The Munnerlyn approach specifies that the ablation pattern is limited to an area referred to as the “optical zone,” or “OZ” where full correction is intended. Surrounding the optical zone is an area referred to as the “transition zone” or “TZ.” The transition zone is added to avoid an abrupt transition in the shape of the cornea at the edge of the optical zone. Prior known transition zone designs have provided continuity of ablation depth but do not guarantee continuity in slope and curvature.

Known formulations of ablation patterns are also algebraically complex in that they result from differences between spherical surfaces. However, spherical surface formulations contain spherical aberrations. In addition, spherical surfaces are not ideal refractive surfaces.

Ablation patterns have been proposed that compensate for induced spherical aberrations using an empirical approach. However, these proposed ablation patterns only correct for spherical myopia and are not useful to correct myopic astigmatism, hyperopia, hyperopic astigmatism, or mixed astigmatism.

Elliptical transition zones for astigmatic ablations have-been proposed which increase the transitional zone for ablations with higher slope transition. This approach limits the slope of the transition zone.

Wavefront-guided laser vision correction procedures have been also used. This procedure measures the aberration of the eye and attempts to correct them. Theoretically, this should decrease the aberration of the eye after surgery. However, it is believed that this procedure results in secondary aberrations greater than the aberration corrected.

### SUMMARY OF THE INVENTION

The present invention relates to a method and apparatus for controlling ablation in refractive surgery using laser ablation patterns to correct refractive errors of the eye such as nearsightedness (myopia), farsightedness (hyperopia), astigmatism, and higher order aberrations of the eye. In accordance with the present invention, post-procedure (also referred to as “post-ablation”) surface smoothing is predicted and the ablation pattern modified to based on the prediction to correct for induced aberrations.

A method is provided for designing laser ablation patterns that corrects refractive errors and compensates for aberrations from post-ablation corneal surface smoothing function. A mathematical model is used to characterize this smoothing function and compensate for post-operative changes to prevent induced optical aberrations. The method also controls positioning of laser pulses to optimize the achievement of the intended ablation pattern.

In accordance with one exemplary embodiment of the present invention, an apparatus is provided for correcting optical aberrations, the apparatus comprising a laser for performing ablation to modify the optical aberrations of the patient's eye, and control means for controlling ablation by the laser in accordance with a control algorithm that corrects for the optical aberrations and further corrects for predicted induced aberrations.

In accordance with a preferred embodiment, the control means includes means for storing a mathematical model that predicts the induced aberrations resulting from surface smoothing, the control means controlling the laser ablation to correct existing refractive error and aberration plus induced aberration predicted by the mathematical model. The adjustment for induced aberration is derived from the mathematical model preferably using a convolution algorithm to predict corneal smoothing. The control algorithm preferably provides a transition zone around an optical zone in accordance with a cubic spline function. The control algorithm preferably corrects myopia and hyperopia by targeting a parabolic-shaped corneal height change. The control means further includes means for storing a rain-drop algorithm to control pulse placement of the laser to control ablation. The control means preferably includes a sorting algorithm to order a laser pulse sequence to minimize the spatial overlap between consecutive laser pulses.

In accordance with another exemplary embodiment of the present invention, a method is provided for modifying refraction of a patient's eye comprising the steps of providing a vision correction laser to modify refraction of the patient's eye, and controlling ablation by said laser in accordance with a control algorithm that corrects for existing measured aberrations and anticipates and corrects for induced aberrations.

In accordance with a preferred embodiment of the method, the step of controlling includes determining a correction map in response to the preoperative measurements, storing a mathematical model that predicts corneal surface smoothing responsive to laser ablation, and determining an ablation map by adjusting the correction map in response to the mathematical model. Preferably, the step of determining the ablation map further includes generating ablation patterns using an iterative deconvolution algorithm that compensates for a predicted surface smoothing response to said ablation. Preferably, the step of controlling further includes the step using a rain-drop algorithm to control pulse placement of said laser to control ablation.

In accordance with yet another exemplary embodiment of the present invention, a computer program product is provided operative in a laser surgical device for correcting optical aberrations, the computer program product comprising a correction mapping stage that determines a correction map based on initial optical measurements, an ablation mapping stage that uses a constrained iterative deconvolution algorithm to compute an ablation map from the correction map, and a pulse control stage using a second iterative algorithm to optimize placement of pulses to avoid spatial overlap.

In accordance with yet another exemplary embodiment of the present invention, a method for correcting optical aberrations comprising the steps of providing a computer controlled ablation device for ablating a patient's eye, performing pre-operative optical measurements to establish a corrective prescription, establishing a mathematical model to predict surface smoothing, establishing an ablation map based on the corrective prescription and the mathematical model, and ablating the patient's eye in accordance with the ablation map.

In accordance with the present invention, ablation patterns or designs compensate for the predicted effects of surface smoothing and thereby improve the accuracy of refractive correction and reduce undesirable secondary or induced aberrations due to corneal healing. The ablation patterns in accordance with the present invention can be used to correct the full range of refractive errors such as myopia (nearsightedness), hyperopia (farsightedness), astigmatism, and higher order aberrations. The method and apparatus of the present invention provides:
1. a mathematical model to predict corneal surface smoothing response to laser ablation;
2. a deconvolution algorithm to generate ablation patterns that pre-compensate for the surface smoothing response;
3. a healing-adjusted ablation pattern including a transition zone around the optical zone for providing continuity in ablation depth and slope;
4. ablation patterns to correct hyperopia, myopia, and astigmatism based on parabolic surfaces in accordance with Zernike polynomials rather than spherocylindrical surfaces (the ideal refractive surface is parabolic);
5. ablation patterns to correct higher-order aberration along with the lower-order refractive errors;
6. an iterative algorithm to generate a sequence of laser pulse placement that produces a result very closely matching the desired ablation map (The sequence has a fractal property such that if it is interrupted at any point, the achieved ablation will still approximate the shape of the complete pattern); and
7. a sorting algorithm to order the pulse sequence to minimize spatial overlap between consecutive or temporally nearby laser pulses.

In accordance with the present invention, a control algorithm produces a more accurate outcome and reduces induced aberrations for the full range of correction of myopia, hyperopia, astigmatism, and higher-order aberration.

### BRIEF DESCRIPTION OF THE DRAWINGS

FIG. 1 depicts an optical zone (“OZ”) surrounded by a transition zone (“TZ”);

FIG. 2A depicts correction and ablation profiles for myopia;

FIG. 2B depicts correction and ablation profiles for hyperopia;

FIG. 3A depicts correction and ablation profiles for myopic astigmatism with flat meridian (−1.50D sphere, −1.00D cylinder×180°);

FIG. 3B depicts correction and ablation profiles for myopic astigmatism with steep meridian (−1.50D sphere, −1.00D cylinder×180°);

FIG. 4A depicts ablation maps for myopia (−1.00D, OZ=6.0 mm, TZ=0.6 mm);

FIG. 4B depicts an ablation map for hyperopia (+1.00D, OZ=5.5 mm, TZ=1.8 mm);

FIG. 4C depicts an ablation map for myopic astigmatism (−1.50D sphere, −1.00D cylinder×180°, OZ=6.0 mm);

FIG. 4D depicts an ablation map for hyperopic astigmatism (+0.00 D sphere, +1.00D cylinder×180°, OZ=5.5 mm, TZ=1.8 mm);

FIG. 5A depicts an ablation pulse map for 1 D of myopia;

FIG. 5B depicts an ablation raw remainder map (the difference between achieved and target ablation maps);

FIG. 5C depicts an ablation residual map after surface smoothing (difference between achieved and target correction maps);

FIG. 6 is a functional block diagram of a laser refractive surgical device in accordance with the present invention;

FIG. 7 is a schematic view of an eye; and

FIGS. 8-11 are flow diagrams of the ablation process in accordance with the present invention;

FIG. 12 depicts an ablation profile for 1 D of hyperopic correction with 5.5 mm diameter optical zone and a 1.8 mm wide transition zone (the achieved surface height change as predicted by the surface smoothing model is shown in dotted line and the difference between the dotted and solid lines is attributed to changes in the epithelial thickness);

FIG. 13 depicts an ablation profile for 1 D of myopic correction with 6.0 mm diameter optical zone;

FIG. 14A depicts a simulation of the ablation pattern for minus-cylinder correction on a EC-5000 with 5.5 mm diameter optical zone and 7.0 mm diameter transition zone with flat meridian;

FIG. 14B depicts a simulation of the ablation pattern for minus-cylinder correction on a EC-500 with 5.5 mm diameter optical zone and 7.0 mm diameter transition zone with steep meridian;

FIG. 15A depicts clinical results of spherical hyperopia correction with slope=−0.708±0.045 (mean±standard error) with intercept set to zero on a LADARVision System (SIRC SE=surgically-induce refractive change in spherical equivalent and Laser SE=laser ablation for spherical equivalent);

FIG. 15B depicts clinical results of spherical myopia correction with slope=−0.968±0.014. (mean±standard error) with intercept set to zero on a LADARVision System (SIRC SE=surgically-induce refractive change in spherical equivalent and Laser SE=laser ablation for spherical equivalent);

FIG. 16A depicts correction/ablation ratios as functions of the smoothing constant s for simulated 1 D ablations of hyperopia;

FIG. 16B depicts correction/ablation ratios as functions of the smoothing constant s for simulated 1 D ablations of myopia;

FIG. 17A depicts an induced spherical aberration (coefficient for Zernike series term Z₄⁰) as functions of the smoothing constant s for simulated 1 D ablations of hyperopia;

FIG. 17B depicts an induced spherical aberration (coefficient for Z₄⁰) as functions of the smoothing constant s for simulated 1 D ablations of myopia;

FIG. 18A depicts model simulation of a 1 D against-the-rule minus cylinder ablation on the EC-5000 (the flat meridian is vertical and the steep meridian is horizontal) showing the ablation-map;

FIG. 18B depicts model simulation of a 1 D against-the-rule minus cylinder ablation on the EC-5000 (the flat meridian is vertical and the steep meridian is horizontal) showing the astigmatism correction/ablation ratio as a function of the smoothing constant s; and

FIG. 18C depicts model simulation of a 1 D against-the-rule minus cylinder ablation on the EC-5000 (the flat meridian is vertical and the steep meridian is horizontal) showing surgically-induced epithelial thickness change with s=0.5 mm.

### DETAILED DESCRIPTION OF INVENTION

Ablation designs, in accordance with the present invention, start with a target corneal surface height change needed to correct preoperative refractive error or aberration. This is referred to as the correction map Δh. The correction map is specified within a central optical zone (“OZ”). The OZ preferably centers on the line of sight and matches the maximum size of the pupil. In accordance with the present invention, the correction map for the correction of myopia, hyperopia, and astigmatism are parabolic. This reduces the induction of aberrations compared with spherical and cylindrical corrections.

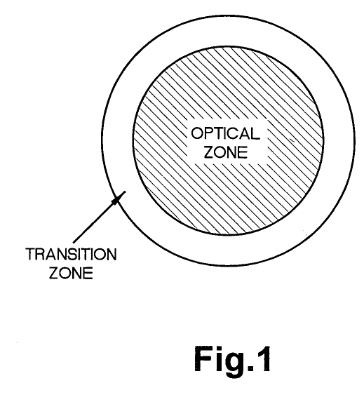

Referring to FIG. 1, the OZ is surrounded by a transition zone (“TZ”) to produce a smooth, continuous blend in ablation depth, slope, and curvature with the surrounding cornea. The OZ and TZ together constitute the entire ablation zone (“AZ”). In accordance with the present invention, a constrained iterative deconvolution algorithm is used to compute the ablation map for the AZ, which, after the expected postoperative surface smoothing effects occur, produces the desired correction in the OZ.

The collection of laser pulses needed to produce the ablation map is then computed in a second iterative algorithm that optimizes the placement of laser pulses. The pulse sequence is then sorted to avoid spatial overlap between consecutive or temporally nearby pulses.

In general, the refractive error correction for an eye must correct for defocus (hyperopia or myopia), astigmatism, and higher order aberrations. The correction maps, in accordance with the present invention, for all three are described below. Further, in accordance with the present invention, these correction maps can be combined to customize the ablation for an individual eye. The ablation map and pulse sequence are then generated using the iterative algorithms.

Correction Map Generation

Correction Map for Defocus

Defocus of the eye is caused by the focusing power of the eye being too strong (myopia or nearsightedness) or too weak (hyperopia or farsightedness). To correct defocus without adding aberration, the ideal corrective lens should be parabolic rather than spherical. Spherical surfaces are associated with spherical aberration. Spherical lens have been historically used in spectacle correction, not for optical superiority, but because traditional lens grinding methods produce spherical surfaces much more readily than aspheric surfaces.

With laser refractive surgery, aspheric ablation can be performed just as easily as spherical patterns. The ablation designs in accordance with the present invention are based on a more optically correct parabolic shape.

Optical surfaces with parabolic shapes are based on the second order of the Zernike circle polynomial series. The Zernike series is a standard for analyzing optical aberration and optical surfaces. In Zernike series, the surface is defined within a circle of unit radius. Each Zernike series term Zₙ^{m} has an order n and an angular frequency m. Defocus (myopia and hyperopia) is described by Z₂⁰ and astigmatism is described by Z₂^{±2}. The preferred correction maps of the present invention for defocus (expressed by Equation 1 below) are defined by the defocus term Z₂⁰(ρ,θ) (expressed by. Equation 2 below).

Defocus error of the eye is conventionally measured by “manifest refraction.” Manifest refraction is performed by trial correction with spherical and cylindrical lenses. The combination of lenses that receive the best subjective rating is the manifest refraction, which can be directly translated to a prescription for spectacle lenses. Spectacle refraction is generally written in notation such as “sphere cylinder×axis.” “Sphere” is the power of the spherical lens, “cylinder” is the power of the cylindrical lens, and “axis” is the orientation of the cylindrical lens. Defocus is measured by the spherical equivalent(“SE”), which is equal to sphere+0.5 cylinder. A formula (expressed by Equation 3 below) is provided for converting the SE to the Zernike coefficient used in Equation 1 below. The corneal index is substituted for the keratometric index.

Defocus can be measured objectively by a wavefront sensor or an autorefractor. With a wavefront deviation measurement, Zernike decomposition is usually determined by analysis software. The defocus coefficient w_{2,0} from the wavefront decomposition specifies the required defocus correction (expressed by Equation 4 below).
Δh_{Defocus}(r,θ)=Δh_{2.0}Z₂⁰(ρ,θ) Equation 1
Z₂⁰(ρ,θ)=\sqrt{3}[2ρ²−1] Equation 2
Δh_{2.0}=−D_{SE}R_{oz}²/[4\sqrt{3}(n−1)] Equation 3
Δh_{2.0}=w_{2.0}/(n−1) Equation 4
where
Δh_{Defocus}(r,θ)=the correction map (target corneal surface height change) in μm,
r=radius from the center of the optical zone in mm, perpendicular to the line of sight,
θ=meridian angle which is zero along +x (to the right when facing the front of the eye) and increases counterclockwise, this follows the right-handed coordinate convention with the height dimension z positive outward along the line of sight,
ρ=r/R_{OZ} is the normalized radius,
R_{OZ}=radius of optical zone in mm,
Δh_{2,0}=coefficient for the Z₂⁰ term of the correction map,
Z₂⁰ (ρ,θ)=Zernike polynomial for defocus,
D_{SE}=spherical equivalent correction in diopter (myopia negative, hyperopia positive) specified at the corneal plane,
n=corneal refractive index, a value of 1.377 is used, and
w_{2.0}=coefficient for the Z₂⁰ term of wavefront deviation.

For myopia correction, the diameter of the OZ is preferably 6.0 mm but adjustable between 5.5 mm and 6.5 mm. Hyperopia correction requires a larger TZ and therefore the OZ has to be relatively smaller to fit inside a reasonable AZ. For hyperopia correction, the diameter of the OZ is preferably 5.5 mm but adjustable between 5.0 mm and 6.0 mm. A smaller OZ may be needed to fit under a smaller LASIK (laser in-situ keratomileusis) flap or to reduce ablation depth in cases of large corrections and thin corneas. A larger OZ may be needed in eyes with larger pupils.

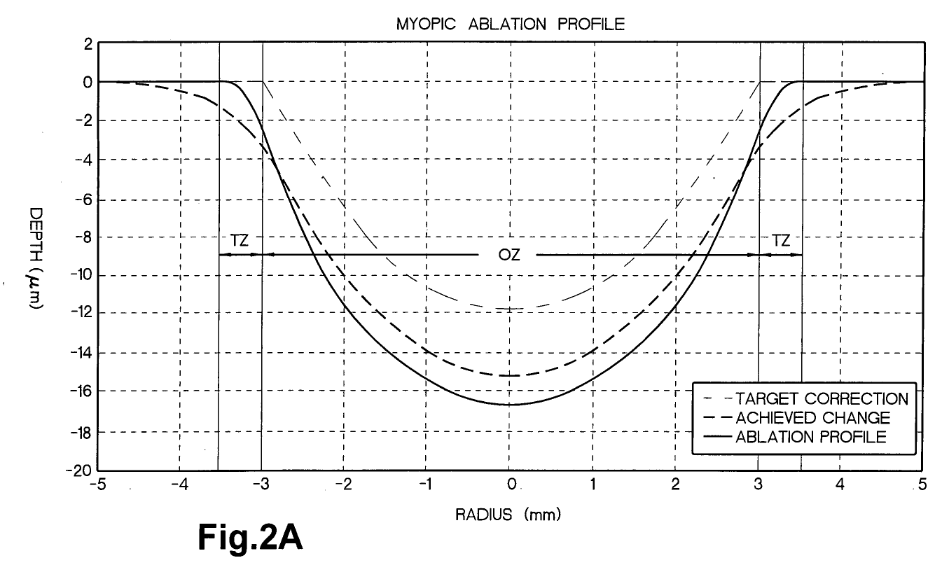

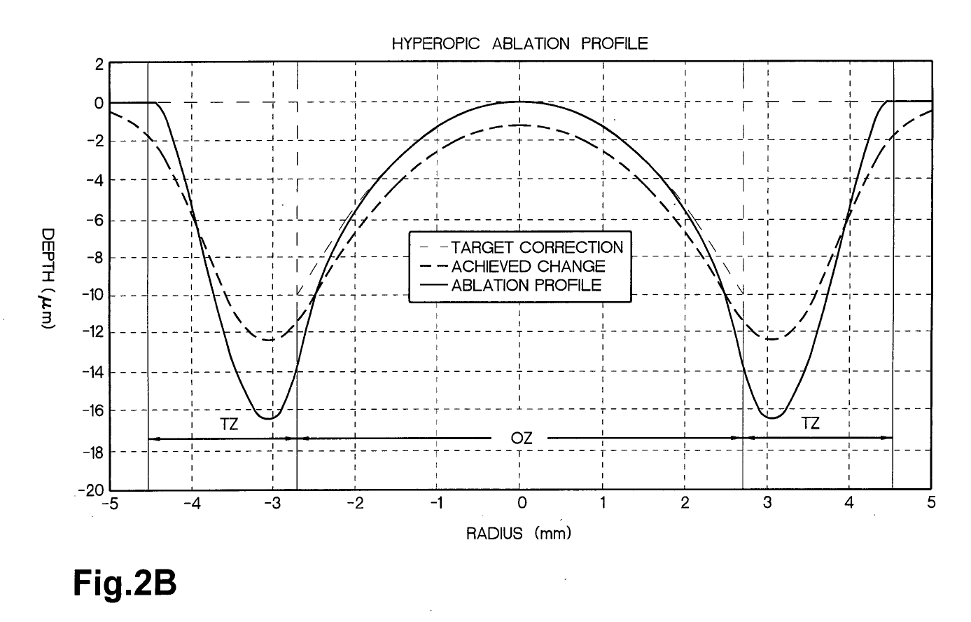

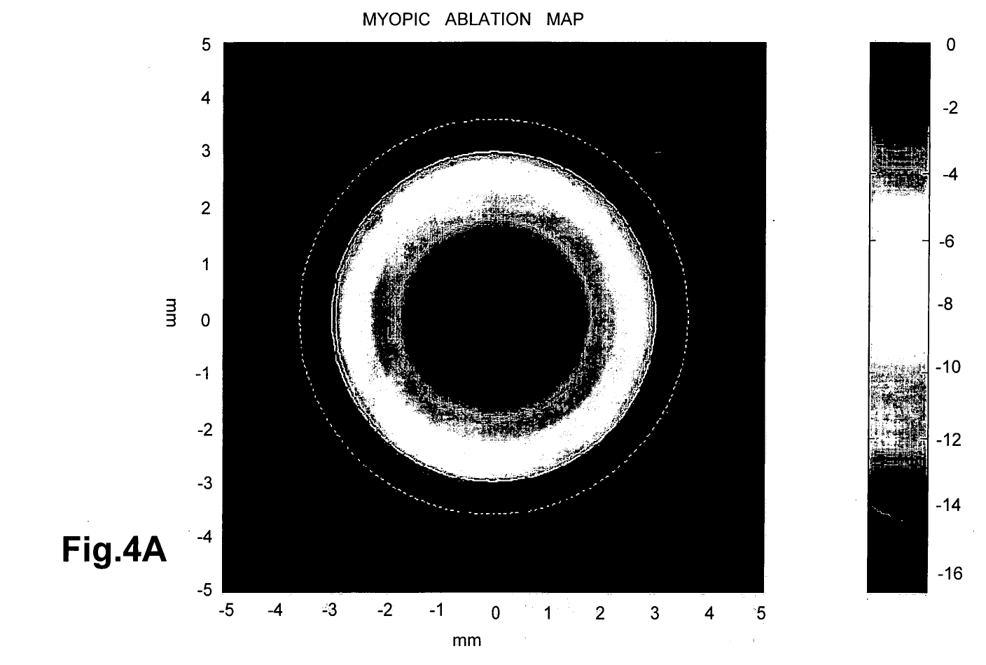

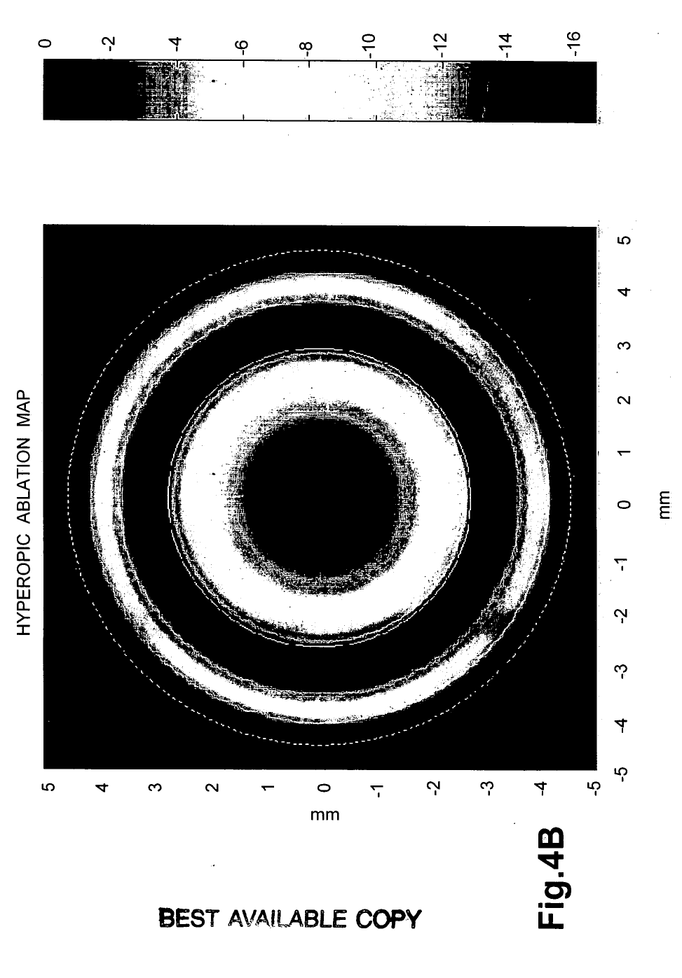

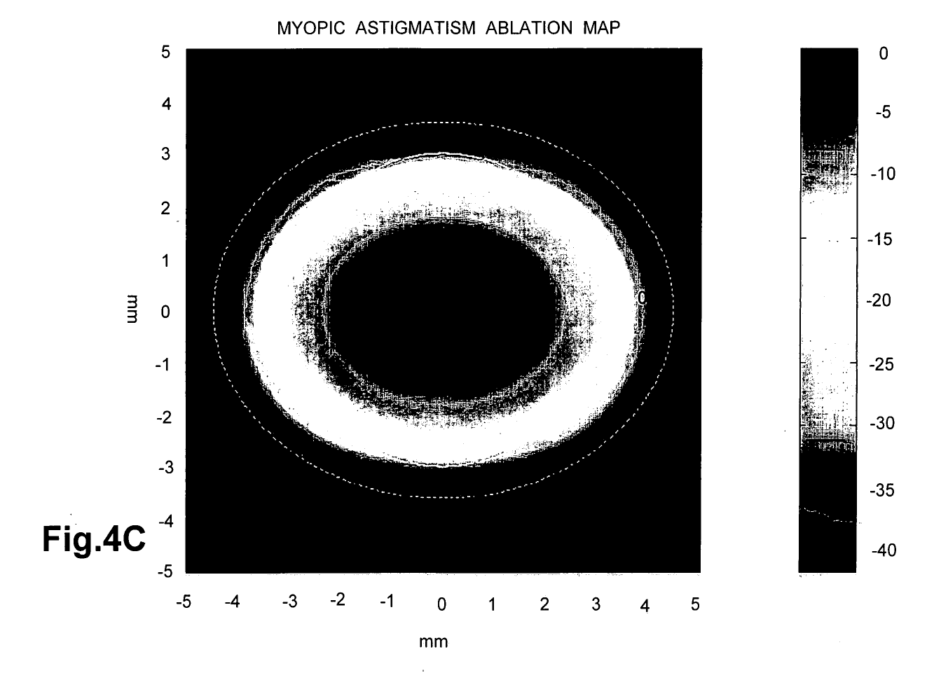

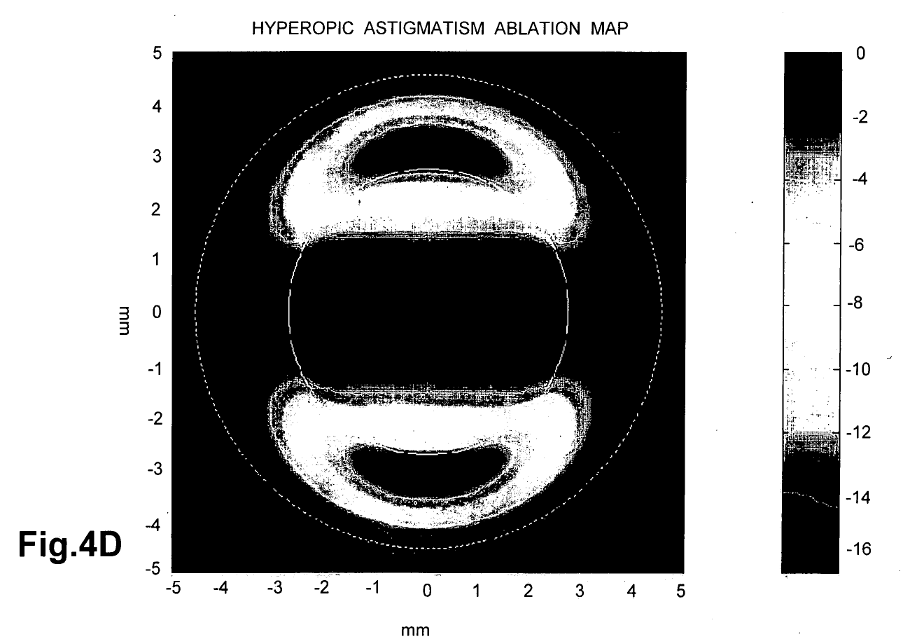

The correction profiles for 1 diopter (“D”) of myopia and hyperopia are shown on FIGS. 2A and 2B, respectively. The corresponding ablations maps are shown on FIGS. 4A and 4B.

Astigmatism Correction Map

The astigmatism correction (expressed by Equation 5 below) is preferably defined with the Zernike terms Z₂²(ρ,Δ) and Z₂⁻²(ρ,Δ), Equation 6 below, which represent cardinal and oblique astigmatism, respectively. The diameter of the OZ is preferably matched to that used for the correction of defocus.

When astigmatism is measured by manifest refraction, Equation 7 below is used to obtain the Zernike coefficients for the correction map. Astigmatism can also be measured by a wavefront sensor, which can be converted to the correction coefficients with Equation 8 below. Finally, if astigmatism is measured by corneal topography, it can be directly translated into the correction coefficients (Equation 9 below).
Δh_{Astig}(r,θ)=Δh_{2,2}Z₂ ²(ρ,θ)+Δh_{2,-2}Z₂⁻² (ρ,θ) Equation 5
Z₂²(ρ,θ)=\sqrt{6}ρ² cos 2θ Equation 6A
Z₂⁻² (ρ,θ)=\sqrt{6}ρ² sin 2θ Equation 6B
Δh_{2,2}=D_{Astig}R_{OZ}² cos(2×axis)/[4\sqrt{6}(n−1)] Equation 7A
Δh_{2,-2}=D_{Astig}R_{OZ}² sin(2×axis)/[4\sqrt{6}(n−1)] Equation 7B
Δh_{2,2}=w_{2,2}/(n−1) Equation 8A
Δh_{2,-2}=w_{2,-2}/(n−1) Equation 8B
Δh_{2,2}=−h_{2,2} Equation 9A
Δh_{2,-2}=−h_{2, -2} Equation 9B
where
Δh_{Astig}(r,θ)=the correction map (target corneal surface height change) in μm,
Z₂²(ρ,θ)=Zernike polynomial for cardinal astigmatism,
Z₂⁻² (ρ,θ)=Zernike polynomial for oblique astigmatism,
Δh_{2,±2}=coefficients for the Z₂^{±2} terms of correction map,
D_{Astig}=astigmatism magnitude in diopters from refraction at the corneal plane,
axis=astigmatism axis,
w_{2,±2}=coefficients for the Z₂^{±2} terms of wavefront deviation, and
h_{2,±2}=coefficients for the Z₂^{±2} terms of corneal topographic height.

Z₂² and Z₂⁻² contain no defocus power and correspond to Jackson cross cylinders (except that the Zernike terms have parabolic profile and the cylinder has circular profile, which is slightly different).

Higher Order Aberration-Correction Map

Higher order aberrations are those that are of more complex shape than defocus and astigmatism. Higher order aberration is described by Zernike terms of order greater than 2. For example, coma is primarily described by Z₃^{±1} and spherical aberration is primarily described by Z₄⁰. Higher order aberration can be measured by either corneal topography or a wavefront sensor. They are separated from lower order aberrations using Zernike series decomposition (expressed by Equation 10 below).
h_{HO}(ρ,θ)=h(ρ,θ)−h_{2,2}Z₂²(ρ,θ)−h_{2,-2}Z₂⁻²(ρ,θ)−h_{2,0}Z₂⁰(ρ,θ)−h_{1,1}Z₁¹(ρ,θ)−h_{1,-1}Z₁⁻¹(ρ,θ)−h_{0,0}Z₀⁰(ρ,θ) Equation 10A
w_{HO}(ρ,θ)=w(ρ,θ)−w_{2,2}Z₂²(ρ,θ)−w_{2,-2}Z₂⁻²(ρ,θ)−w_{2,0}Z₂⁰(ρ,θ)−w_{1,1}Z₁¹(ρ,θ)−w_{1,-1}Z₁⁻¹(ρ,θ)−w_{0,0}Z₀⁰(ρ,θ) Equation 10B
where
h(ρ,θ) is the corneal topographic height,
subscript _{HO} denotes sum of higher-order terms,
ρ=r/R_{OZ}, the normalized radius,
h_{n,m} are the coefficients from Zernike decomposition of h(ρ,θ),
w(ρ,θ) is the wavefront height, and
w_{n,m} are the coefficients from Zernike decomposition of w(ρ,θ).

The correction map Δh_{HO}(r,θ) for higher order aberration is described by Equation 11 (topography-guided) or Equation 12 below(wavefront-guided).
Δh_{HO}(r,θ)=−h_{HO}(r,θ) Equation 11
Δh_{HO}(r,θ)=w_{HO}(r,θ)/(n−1) Equation 12
Combining Correction Maps

The correction maps for defocus and astigmatism are combined to produce the target refractive correction. If higher order aberration has been measured by a wavefront sensor or corneal topography system, it can also be added to the target correction map at this step. All of the component maps must have the same OZ diameter. The combination is described in Equation 13A below. The maximum point of the correction map is adjusted to zero in Equation 13B below.
Δhₜₑₘₚ(r,θ)=Δh_{Defocus}(r,θ)+Δh_{Astig}(r,θ)+Δh_{HO}(r,θ) for r<R_{OZ} Equation 13A
Δh_{target}(r,θ)=Δhₜₑₘₚ(r,θ)−max{Δhₜₑₘₚ(r,θ)} for r<R_{OZ} Equation 13B
where
Δhₜₑₘₚ(r,θ) is the combined target correction map before adjustment,
R_{OZ} is the optical zone radius in global coordinates,
Δh_{target}(r,θ) is the final combined target correction map, and
max{ } returns the maximum height of the enclosed surface.
   Ablation Map Generation with Iterative Deconvolution Epithelial Smoothing Model

The corneal surface change specified by Δh_{target}(x,y) will be different from the ablation map a(x,y) because of a corneal surface smoothing response. One mechanism of smoothing is the migration of surface epithelial cells away from more convex areas (islands) and into less convex areas (divots). In accordance with the present invention, this smoothing response can be represented by a mathematical model that predicts this smoothing response. Details regarding construction of the mathematical model to predict the smoothing response in accordance with the present invention are described below in detail under the section entitled “Construction of Mathematical Model of Corneal Surface Smoothing after Laser Refractive Surgery.”

According to the mathematical model of the present invention, Δh′ (x,y) relates to a′ (x,y) by a convolution operation (expressed by Equation 14A below) with smoothing function f′ (x′,y′) (expressed by Equation 14C below).
Δh′(x′,y′)=a′(x′,y′)\mathring{×}f′(x′,y′) Equation 14A
ΔH′(ωₓ′,ω_{y}′)=A′(ωₓ′,ω_{y}′)F′(ωₓ′,ω_{y}′) Equation 14B
F′(ωₓ′,ω_{y}′)=1/[1+s²(ωₓ′²+ω_{y}′²)] Equation 14C
where
Δh′ (x′,y′) is the corneal surface height change in local coordinates,
a′(x′,y′) is the ablation map in local coordinates,
f′(x′,y′) is the impulse response function of the corneal surface (convolution with f′ (x′,y′) describes corneal surface smoothing, f′ (x′,y′) is the inverse Fourier transform of F′ (ωₓ′, ω_{y}′)),
ΔH′ (ωₓ′, ω_{y}′) is the Fourier transform of Δh′ (x′,y′),
ωₓ′ and ω_{y}′ are the respective spatial frequencies for x′ and y′ in radians/length,
A′ (ωₓ′, ω_{y}′) is the Fourier transform of a′ (x′,y′)
F′ (ωₓ′, ω_{y}′) is frequency response of the corneal surface (it's the Fourier Transform of f′ (x′,y′)), and
s is the smoothing constant that characterizes the epithelial smoothing model (1/s is the cutoff frequency (radian/length) of the low-pass filter (Equation 14C)).

Equations 14A and 14B are equivalent expressions of the corneal surface smoothing model in the spatial and frequency domains, respectively. The spatial and frequency domains are related by the Fourier transform. The frequency response of the corneal surface (Equation 14C) has the form of two first-order Butterworth low-pass filters in series. The filter is characterized by a smoothing constant s. Clinical data from laser in-situ keratomileusis (LASIK) has been used to estimate the value of s. The best-fit values for corrections of myopia, myopic astigmatism, and hyperopia have been found to range between 0.32 and 0.63 mm. The average was 0.5 mm. According to these results, s=0.5 mm is preferably used to generate the ablation map in the deconvolution algorithm described below. However, the control algorithm of the present invention is not restricted to a specific value of smoothing constant.

Conversion Between Local and Global Coordinate Systems

Equations 14A-C are expressed in local coordinate system x′,y′ which are tangential to the corneal surface. In the local coordinate system, z′ is defined as being perpendicular to the local corneal surface. The local coordinate system has the perspective of a small epithelial cell on the corneal surface and is important to the development of the epithelial smoothing theory. The corneal height and ablation depth are preferably specified in fixed global coordinate system x, y, z. In the global coordinate system, z is defined as the line of sight. The global coordinate system has the perspective of the laser scanning system looking down at the cornea. The coordinate systems are identical at the corneal apex but the deviation increases as the radial distance approaches the corneal radius of curvature.

The following approximate coordinate conversions are used to apply the smoothing theory (Equations 14A-C) to ablation design. The equations treat the corneal surface as a perturbation from a sphere.
a′(r′,θ)=a(r,θ)cos[arcsin(r/R_{C})] Equation 15A
a(r,θ)=a′(r′,θ)/cos(r′/R_{C}) Equation 15B
r′=R_{C} arcsin(r/R_{C}) Equation 15C
r=R_{C} sin(r′/R_{C}) Equation 15D
Δh′(r′,θ)=Δh(r,θ)cos[arcsin(r/R_{C})] Equation 15E
Δh(r,θ)=Δh′(r′,θ)/cos(r′/R_{C}) Equation 15F
where
r′ is the local radial coordinate equivalent to the arc length from the corneal apex (the apex is defined as the intersection between the line of sight and the anterior corneal surface);
r is the global radial coordinate, the radial distance measured perpendicularly from the line of sight (the line of sight is defined as the line passing through the center of the eye's entrance and exit pupils connecting the object of regard to the foveola);
R_{C} is the radius of curvature of the corneal best-fit sphere (a value of 7.6 mm is used in simulations);
global height quantities h, Δh, and a are measured along the z axis, which is positive outward along the line of sight; and
θ is the meridian angle or the azimuth, which is zero on the +x axis and increases counterclockwise.
   Conversion Between Polar and Cartesian Coordinate Systems

The coordinate transforms in Equations 15A-F are performed in polar coordinates. To convert to and from the Cartesian coordinate system, the following equations are used.
x=r cos θ Equation 16A
y=r sin θ Equation 16B
r=\sqrt{(x²+y²)} Equation 16C
θ=arctan(y/x) Equation 16D
and
x′=r′ cos θ Equation 17A
y′=r′ sin θ Equation 17B
r′={square root over (x′²+y′²))} Equation 17C
θ=arctan(y′/x′) Equation 17D
where
x is perpendicular to the line of sight and positive to the right of a viewer facing the eye; and
y is perpendicular to the line of sight and positive upward (with face upright).
   Deconvolution to Compute Expected Smoothing

The ablation pattern that compensates for the expected smoothing change can be computed by deconvolution. Deconvolution is the reverse of the convolution operation shown in Equation 14A. It can be written in the frequency domain as:
A′(ωₓ′,ω_{y}′)=ΔH′(ωₓ′,ω_{y}′)/F′(ωₓ′,ω_{y}′) Equation 18

Equation 18 is generally not solved directly because F′ approaches zero at high spatial frequencies. Division by zero is not defined. Division by a very small number is computationally susceptible to noise or rounding errors. Stable solution is usually obtained by constrained iterative deconvolution where the smoothing change described by Equation 14A is subtracted from the ablation pattern until the ideal ablation map converges.

Transition Zone Width Determination

The surface smoothing model, in accordance with the present invention, indicates that any sudden change in depth or slope on the corneal surface would be smoothed over. To obtain the desired surface change in the optical zone, it must be surrounded with a gradual transition. The transition zone occupies the area between the borders of the ablation zone and the optical zone.
a′(r′,θ)=spline′(r′,θ) for R_{AZ}′(θ)>r′>R_{OZ} Equation 19A
W_{TZ}′(θ)=R_{AZ}′(θ)−R_{OZ}′ Equation 19B
W_{TZ}(θ)=R_{AZ}(θ)−R_{OZ} Equation 19C
where
spline′ (r′,θ) is the spline function for the TZ,
R_{OZ}′ is the optical zone radius in local polar coordinates,
R_{AZ}′ (θ) is the ablation zone radius in local-polar coordinates,
W_{TZ}′ (θ) is the transition zone width in local polar coordinates,
R_{OZ} is the optical zone radius in global polar coordinates, and
R_{AZ}(θ) is the ablation zone radius in global polar coordinates, and
W_{TZ}(θ) is the transition zone width in global polar coordinates.

The width W_{TZ}′ of the transition zone (TZ) is preferably set so that the transition spline profile has component frequencies mostly below that of the cutoff radial frequency 1/s radian/mm. Hyperopic correction profiles need a wider transition zone because the transition contains more phases (FIG. 2B) compared to a myopic correction (FIG. 2A). The transition zone for the myopia correction profile (FIG. 2A) goes through roughly ¼ cycle of a sinusoid (π/2 radian). The transition zone for the hyperopia correction profile (FIG. 2B) goes through roughly ¾ cycle of a sinusoid (3π/2 radian). Therefore, W_{TZ}′ should be approximately 1/2 π s for myopia and 1.5 π s for hyperopia correction. For s=0.5 mm, W_{TZ}′=½ π s translates to W_{TZ}′=0.8 mm for myopia correction. Converted into global coordinates, W_{TZ}=0.6 mm. Proportionally, the preferred W_{TZ}=1.8 mm for hyperopia corrections.

The TZ for myopic astigmatism is more complicated. On the steep meridian, the TZ is identical to that for myopia and W_{TZ}=0.6 mm is used. On the flat meridian, the TZ profile gains more phase with increasing ratio of astigmatism to myopia. Therefore, the preferred shape of the AZ is elliptical and the width of the TZ depends on the meridian. The widths on the flat and steep meridians determine the diameters of the elliptical AZ on these cardinal meridians. The diameters for the AZ and the TZ widths can then be calculated on all meridians using the equations for an ellipse.

For abs (D_{SE})>0.5 D_{Astig} and D_{SE}<0 (myopic astigmatism)
W_{TZ}_{_{—}}ₛₜₑₑₚ=0.6 mm, Equation 20A1
W_{TZ}_{_{—}}_{flat}=W_{TZ}_{_{—}}ₛₜₑₑₚ+min(1.8,R_{OZ}{sqrt[(−D_{SE}+0.5D_{Astig})/−D_{SE}−0.5D_{Astig})]−1}) Equation 20A2
where
min ( ) returns the minimum member of the input set,
abs ( ) is the absolute value function,
sqrt ( ) is square root function,
D_{SE}=spherical equivalent correction in diopter (myopia negative, hyperopia positive) specified at the corneal plane, and
D_{Astig}=astigmatism magnitude in diopters (positive) from refraction at the corneal plane.

For mixed astigmatism, the flat meridian has a profile similar to that for hyperopia and a 1.8 mm TZ width is preferred. The transition profile on the steep meridian depends on the SE. Therefore, the preferred AZ shape is again elliptical. In a preferred system, the steep meridian's TZ width transitions from 0.6 mm to 1.8 mm, depending on the ratio of SE to astigmatism, according to Equations 20B1-2.

For abs (D_{SE})<0.5 D_{Astig} (mixed astigmatism)
W_{TZ}_{_{—}}ₛₜₑₑₚ=1.2+(1.2D_{SE}/D_{Astig}) Equation 20B1
W_{TZ}_{_{—}}_{flat}=1.8 mm Equation 20B2

For mixed astigmatism, all meridians have profiles similar to that for hyperopia and a 1.8 mm TZ width is preferred.

For abs (D_{SE})>0.5 D_{Astig} and D_{SE}>0 (hyperopic astigmatism)
W_{TZ}_{_{—}}ₛₜₑₑₚ=1.8 mm Equation 20C1
W_{TZ}_{_{—}}_{flat}=1.8 mm Equation 20C2

In most cases, the TZ width does not need to be adjusted for higher order aberration because it is generally much smaller in magnitude relative to the coexisting defocus and astigmatism. However, if the higher order aberration is larger than the defocus and astigmatism, then the larger 1.8 mm TZ width is preferred all around.

Cubic Spline in the Transition Zone

A cubic spline curve in the TZ that provides a continuous transition in depth and radial slope is preferred. Discontinuity in depth and slope provokes large healing responses (epithelial hyperoplasia, subepithelial haze) and cause tear film instability.

The cubic spline function is a 3^{th} order polynomial in r′ (Equation 21A below). Its first partial derivative with respect to r′ is the radial slope (Equation 21B below). Its second partial-derivative with respect to r′ is the radial curvature (Equations 20C above).
spline′(r′,θ)=c₀(θ)+c₁(θ)r′+c₂(θ)r′²+c₃(θ)r′³ Equation 21A
∂spline′(r′,θ)/∂r′=c₁(θ)+2c₂(θ)r′+3c₃(θ)r′² Equation 21B

The spline coefficients cₙ(θ) are solved at each meridian θ with the boundary conditions at the edge of the ablation zone r′=R_{AZ}′ and at the edge of the optical zone r′=R_{OZ}′
c₀(θ)+c₁(θ)R_{AZ}′+c₂(θ)R_{AZ}′²+c₃(θ)R_{AZ}′³=0 Equation 22A
c₁(θ)+2c₂(θ)R_{AZ}′+3c₃(θ)R_{AZ}′²=0 Equation 22B
c₀(θ)+c₁(θ)R_{OZ}′+c₂(θ)R_{OZ}′²+c₃(θ)R_{OZ}′³=a′(R_{OZ}′, θ) Equation 23A
c₁(θ)+2c₂(θ)R_{OZ}′+3c₃(θ)R_{OZ}′²=b′(R_{OZ}′,θ) Equation 23B
where
b′ (r′,θ)=∂a′ (r′,θ)/∂r′ is the radial slope of the ablation profile in local polar coordinates.

Equations 22 and 23 form a system of 4 linear algebraic equations with 4 unknowns cₙ(θ). These equations are solved using well known linear algebra techniques.

Depth Constant for Non-Positivity Constraint

The ablation depth, in accordance with the present convention, is non-positive. A negative number corresponds to tissue removal. A positive number corresponds to tissue addition, which is not possible with laser ablation. To enforce this non-positivity constraint, a constant depth offset d is added to the entire ablation map. d is computed in two steps.
d=d₁+d₂ Equation 24
where
d₁+d₂ are the two components of d.
   d₁=−maximum{a₀′(r′,θ)] for r′<R_{OZ}′ Equation 25A
   a₁′(r′,θ)=a₀′(r′,θ)+d₁ for r′<R_{OZ}′ Equation 25B
   where
maximum{ } is a function that returns the maximum value of the enclosed function, and
a₀′ (r′,θ) is the ablation map without any constant offset.

Equation 25A computes the minimum depth offset to keep the OZ non-positive.
d₂=−maximum{(⅓)b₁′(R_{OZ}′,θ)W_{TZ}′+a₁′(R_{OZ}′,θ)} Equation 26A
a′(r′,θ)=a₁′(r′,θ)+d₂ for r′<R_{OZ}′ Equation 26B
where
b₁′(r′,θ)=∂a₁′(r′,θ)/∂r′ is the radial slope of the ablation profile in local polar coordinates.

Equation 26A computes the minimum additional depth offset required to keep the TZ non-positive when there is an upward radial slope at the edge of the OZ.

Iterative Deconvolution Loop

Iterative deconvolution is used to generate the ablation map. The ablation map is initially set to be the same as the correction map inside the OZ. The correction map Δh_{target}(r,θ) specifies the desired corneal surface change (Equation 13). It is converted to the local-coordinate form for the deconvolution computations.
a₀′[0](r′,θ)=Δh′_{target}(r,θ) for r′<R_{OZ}′ Equation 27
where
a₀′[0] (r′,θ) is the initial ablation map (the bracket [0] denotes the 0^{th} iteration).

The depth offset is then computed according to Equations 25A-26B and the transition zone map is computed according to Equations 19A-23B to yield the initial ablation map a′ [0] (r′,θ).

The corneal surface smoothing is then simulated by convolution (Equation 14). This is done in Cartesian coordinates.
Δh′[i](x′,y′)=a′[i−1](x′,y′)\mathring{×}f′(x′,y′) Equation 28
where the iteration index bracket [i] denote the ith iteration. The loop starts with i=1.

The smoothing effect is canceled out by adjusting the ablation map. This is done in polar coordinates.
g′[i](r′,θ)=Δh′[i](r′,θ)−Δh′_{target}(r′,θ) for r′<R_{OZ}′ Equation 29 A
a₀′[i+1](r′,θ)=a₀′[i]((r′,θ)−g′[i](r′,θ) for r′<R_{OZ}′ Equation 29B
where g′[i] (r′,θ) is the difference map between the outcome map Δh′[i] (r′,θ) and the target correction map Δh′_{target}(r′,θ).

Again, the depth offset is then computed according to Equations 25A-26B and the transition zone map is computed according to Equations 19A-23B to yield the compute initial ablation map a′ [i+1] (r′,θ). The loop is then repeated and the iteration index is incremented. The loop ends when the outcome and target corrections have the same shape and the difference map approaches a constant value.
δ=maximum{g′[i](r′,θ)}−minimum{g′[i](r′,θ)} for r′<R_{OZ}′ Equation 30A
δ<δₘₐₓ Inequality 30B

Inequality 30B is the termination condition for the iteration loop. The maximum tolerable difference δₘₐₓ is a small depth, such as 0.1 μm, that is smaller than the resolution of the ablation process. The final iteration yields an ablation map a′ (r′θ) that is very close to the ideal needed to produce target correction. The ablation map is converted to the global Cartesian coordinate (Equations 15A-16D) for the next step, where the laser pulse sequence is calculated.

The above algorithm is used to generate ablation maps for myopia (FIG. 4A), hyperopia (FIG. 4B), myopic astigmatism (FIG. 4C) and hyperopic astigmatism (FIG. 4D). The smoothing constant s=0.5 mm was used. For myopia, the deconvolution deepens the ablation in the OZ. This is caused by steepening of the ablation slope in the peripheral OZ (FIG. 2A, 4A). The algorithm, in accordance with the present invention, compensates for the healing response after any correction pattern, including myopia, hyperopia, astigmatism, and higher-order aberration.

For hyperopia correction, the deconvolution also deepens the ablation in the OZ, more in the periphery than at the center (FIG. 2B, 4B). The hyperopic ablation map, in accordance with the present invention, puts the deepest ablation outside of the OZ. This is significantly different from conventional ablation algorithms, which place the deepest ablation at the edge of the OZ. The deconvolution algorithm, in accordance with the present invention, guarantees accurate full correction in the OZ and extends some correction into the transition zone. In contrast, conventional hyperopic ablation achieves no correction in the TZ and produces full correction only in the center of the OZ, with gradual decrease in correction toward the peripheral OZ.

To achieve the improvement in optical quality in the OZ, the ablation patterns, in accordance with the present invention, require more ablation depth than conventional algorithms. The ablation depths per diopter for the correction of myopia and hyperopia are tabulated for a range of OZ diameters in Table 1. The depth is measured at the deepest point of the ablation.

TABLE 1
Ablation depth (μm) per diopter, measured at the
deepest point of the ablation map.

| OZ size | 5 mm | 5.5 mm | 6 mm | 6.5 mm |
|---|---|---|---|---|
| Myopia | 12.4 | 14.4 | 16.6 | 18.8 |
| Hyperopia | 14.4 | 16.6 | 19.1 | NA |

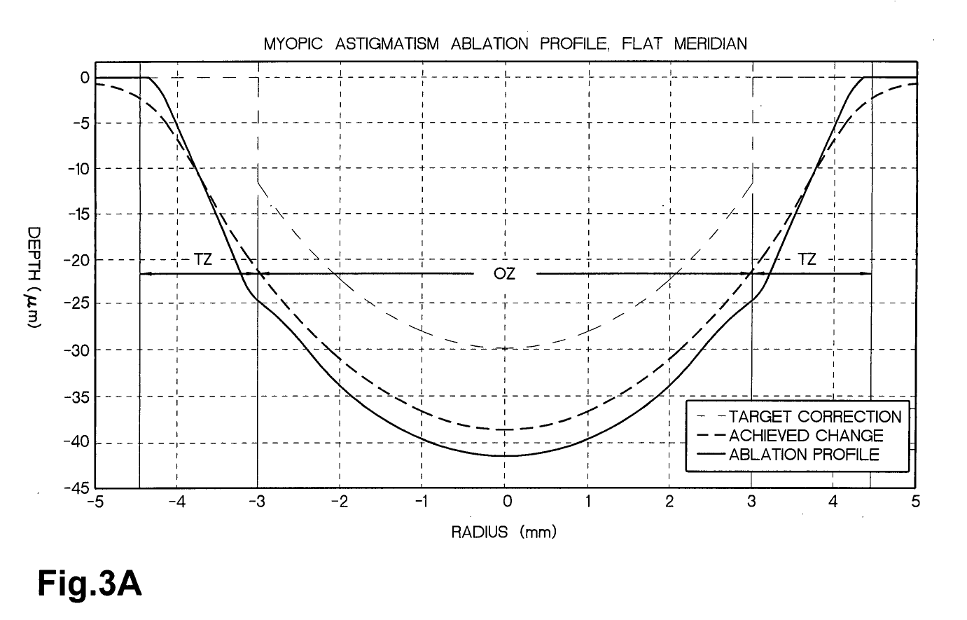

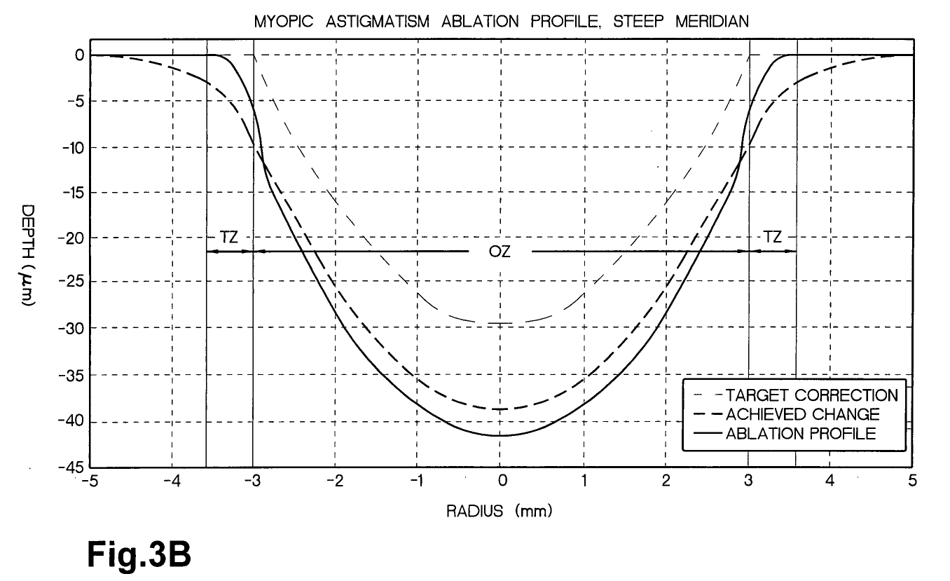

The myopic astigmatism ablation map in accordance with the present invention has an elliptical AZ (FIG. 4C) and a circular OZ. The eccentricity of the ellipse is determined by the ratio of astigmatism to myopia (Equations 20). The ablation profile on the steep meridian is similar to that for pure myopia (FIG. 3B). On the flat meridian, the ablation is deeper at the edge of the OZ to compensate for the expected epithelial hyperplasia at that location (FIG. 3A).

The hyperopic astigmatism ablation profile has the same shape as that for hyperopia, but the depth of ablation varies from meridian to meridian (FIG. 4D).

Pulse Sequence Generation

Laser systems produce ablation patterns by scanning laser pulses on the cornea. Thus, the final instruction to the laser system consists of a sequence of locations for the placement of pulses. In accordance with the present invention, the ablation maps are translated into pulse placement sequences with a “rain-drop” algorithm. An optional sorting algorithm is then used to order the pulses so that temporally nearby pulses do not overlap spatially.

The simplest method of translating an ablation map to a pulse map is to apply pulses with a density proportional to the ablation map a(x,y). This approach leads to a distortion of the ablation caused by the smearing effect of the laser spot. The larger the spots used, the more the outcome deviates from the target ablation. The distorted outcome can be simulated by convolving the target ablation map with the laser spot map. To reduce the distortion, a “rain-drop” algorithm is used to plan the pulse map. The rain-drop algorithm is a constrained iterative deconvolution procedure, like the algorithm used to generate the ablation map. A major difference is that the pulse map necessarily consists of discrete impulses to represent the pulses, whereas the ablation map is continuous. Another difference is that the pulse map is linked to a pulse sequence that has an additional temporal dimension. The rain-drop algorithm introduces a random element to give the sequence a desirable fractal property.

Rain-Drop Algorithm

The rain-drop algorithm generates randomly located pulses. Whether the pulses are rejected or lands on the simulation grid depends on several constraints and a probability test. The constraints are:
1. Non-coincidence. Pulses cannot fall on the exact same location twice. This condition is not essential, but helps reduce deep edges, which is an issue with flat-top beam profiles.
2. Non-positivity. Pulses cannot fall where there is positive value within the spot area.
3. Border. The entire spot must fit inside the AZ borders.
   If a pulse passes the constraints, a probability test is applied to determine if the pulse is finally accepted. The probability of acceptance is proportional to the average depth of the remainder map inside the pulse spot. The averaging may be weighed by the pulse map (more accurate) or weighed uniformly (faster computation). The remainder map is the difference between the achieved ablation and the target ablation map.

The rain-drop algorithm can be applied to a situation where more than one spot size is used. The largest spot is used first. The sequence switches to the next smaller spot size after a preset number of larger spots are rejected by the constraints and probability test. After the random rain-drop algorithm runs through all the spot sizes, a deterministic fill-in phase is used to fill in the remaining low spots on the remainder map. The fill-in algorithm only uses the smallest spot size. The constraints are not applied in the fill-in phase. The remainder map is convolved with the spot map to obtain a guide map. Pulses are applied to the lowest spot on the guide map until the average level of the remainder map reaches zero.

The random nature of the rain-drop algorithm gives the generated pulse sequence a fractal distribution. Fractal patterns are self-similar at different size scales. Fractal distribution means that the pulse sequence is ordered in such a way so that if the sequence is stopped at any point, the fractional correction has approximately the same shape as the complete target correction. This property also makes the shape of the overall correction less susceptible to temporal variations in pulse energy, tissue hydration, or any other aspect of the ablation process.

The rain-drop algorithm is detailed in the following program code written in the style of C programming language.

/* RAINDROP ALGORITHM TO GENERATE PULSE MAP & SEQUENCE */

| | |
|---|---|
| int i, j; | /* indices for sequence & loops */ |
| int ix, iy; | /* indices for map arrays */ |
| int ix_start, iy_start; | /* starting indices for the ablation zone */ |

float target_volume, mean_level, bottom_depth, az_area, az_width, az_height;

| | |
|---|---|
| float spot_diameter[N_SPOT_SIZES]; | /* spot sizes (diameters) */ |
| float spot_volume[N_SPOT_SIZES]; | /* ablation volume per pulse for each spot size */ |

| | |
|---|---|
| float spot_map[N_SPOT_SIZES][NX][NY]; | /* ablation maps for a single pulse */ |

/* NX, and NY, are the number of elements in the x and y dimensions of the simulation grid */

| | |
|---|---|
| float pulse_map[N_SPOT_SIZES} [NX][NY]; | /* one pulse map for each spot size */ |
| float remainder[NX][NY]; | /* remainder map */ |
| float guide_map[NX][NY]; | |
| float target_ablation[NX][NY]; | /* the total target ablation map */ |
| struct Location_indices {int ix, iy;} | /* structure consisting of x, y indices */ |
| Location_indices min_ixy | /* location indices of the minimum value on the map */ |
| struct Location {float x, y;} | /* structure consisting of x, y coordinates */ |

| | |
|---|---|
| Location sequence[N_SPOT_SIZES][MAX_PULSES] | /* Pulse sequences */ |

| | |
|---|---|
| int N[N_SPOT_SIZES]; | /* the number of pulses for each fraction of pulse map */ |

int rejection, rejection_flag, max_rejection;
/* initially, remainder map = ablation map */
equate_map(remainder, target_ablation);
/* equate_map(map1, map2) performs the array function map1 = map2 */
target_volume = integrate_volume(target_ablation);
/* calculate the volume (negative) of the remainder map */

| | |
|---|---|
| mean_level = target_volume / az_area; | /* az_area is the area of the ablation zone */ |
| bottom_depth = min_map(remainder); | /* returns the most negative value on remainder map */ |

/* random “rain-drop” stage */
for (i=0; i < N_SPOT_SIZES−1; i++) {

| | |
|---|---|
| make_spot_map(spot_map[i], spot_diameter[i]); | /* generate spot map */ |
| spot_volume[i] = integrate_volume(spot_map[i]); | /* spot volume is negative */ |

| | |
|---|---|
| j = 0; | /* sequence count reset to zero */ |
| rejection = 0; | /* consecutive rejection count */ |

max_rejection = az_area/(PI*(spot_diameter[i]/2)\hat{ }2) + 100;
while (rejection < max_rejection) {

| /* random( ) generate random value between 0 to 1 */ | |
|---|---|
| /* DX and DY are grid element sizes along horizontal and vertical dimensions */ | |
| /* ix_start and iy_start are the lowest x,y indices where the AZ starts */ | |
| /* az_width and az_height are the horizontal and vertical diameters of the AZ */ | |
| ix = ix_start + round(random( )*az_width/DX); | /* round(float) returns the closest integer |

*/

iy = iy_start + round(random( )*az_height/DY);
rejection_flag = 1;
if (pulse_map[i][ix][iy] == 0)
/* pulse cannot fall on same place twice */
if (inside_AZ(ix,iy,spot_diameter[i] /2) == TRUE) {
/* pulse must not ablate outside the ablation zone */

if (spot_max(remainder ix, iy, spot_diameter[i]) < 0)
/* spot_max( ) return the maximum value inside the pulse spot diameter.
Pulse is rejected if remainder level above zero anywhere inside spot. */
if (spot_mean(remainder,ix,iy,spot_diameter[i]) / bottom_depth > random( )) {
/* spot_mean( ) calculate the mean depth inside the pulse spot diameter */

sequence[i][j++] = locate(ix, iy);
/* the location x,y corresponding to indice ix,iy is added to the pulse sequence */
pulse_map[i][ix][iy] = 1;
ablate(remainder, spot_map[i], ix, iy);
/* ablate(Map,Spot,ix,iy) subtract Spot at Location_indices ix,iy from Map */
mean_level −= spot_volume[i] / az_area;
bottom_depth = min_map(remainder);
rejection_flag = 0;
rejection = 0;
}

if (rejection_flag = 1) rejection ++;
}}

N[i] = j;
}
/* deterministic fill-in stage for the last layer of the last spot size (smallest) */
i--;
while (mean_level < 0) {

guide_map = convolve(spot_map[i], remainder);
min_ixy = min_location_indices(guide_map);
/* min_location_indices(map) returns the x,y indices of the minimum value on the map */
sequence[i][j++] = locate(min_ixy.ix, min_ixy.iy);
pulse_map[i][min_ixy,ix][min_ixy.iy] += 1;
ablate(remainder, spot_map[i], min_xy.ix, min_xy.iy);
mean_level −= spot_volume[i] / az_area;
}

N[i] = j;
/* END ALGORITHM */

Because the rain-drop algorithm constantly updates the remainder map to guide subsequent pulse placement, the final remainder map is very close to flat zero. The accuracy of the algorithm is demonstrated using the following examples.

For the examples, a Gaussian beam fluence profile is used defined in the following equation.
G(r)=F₀ exp{−r²/R_{beam}²} Equation 31
where
R_{beam} is the 1/e beam radius,
exp{ } is the natural exponential function with base e, and
F₀ is the peak fluence.

The ablation profile is calculated from the beam fluence using a Beer's law approximation of the ablation process. The ablations characteristics of the 193 nm wavelength ArF excimer laser were used.
S(x,y)=E ln{G(r)/Fₜₕ)} for G(r)>Fₜₕ Equation 32
S(x,y)=0 for G(r)≦Fₜₕ
where
S (x,y) is the ablation depth map from a single laser pulse (“spot map”),
ln{ } is the natural logarithm function with base e,
E is the ablation efficiency, and
Fₜₕ is the threshold fluence for ablation.

The spot diameter is defined as the diameter inside which S(x,y)>0.

For our simulations, an ablation efficiency of 0.3 micron and a threshold fluence of 60 mJ/cm² are adopted.

For the examples, a peak fluence F₀=e Fₜₕ was used. This provides a central ablation depth of 0.3 microns and ablation spot radius Rₛₚₒₜ=R_{beam}. Ablation spot diameters of 2.0 mm and 1.0 mm were used. The rationale for using two spot diameters is to use the larger spot to remove most of the required volume quickly, then use the smaller spot to achieve a higher final accuracy.

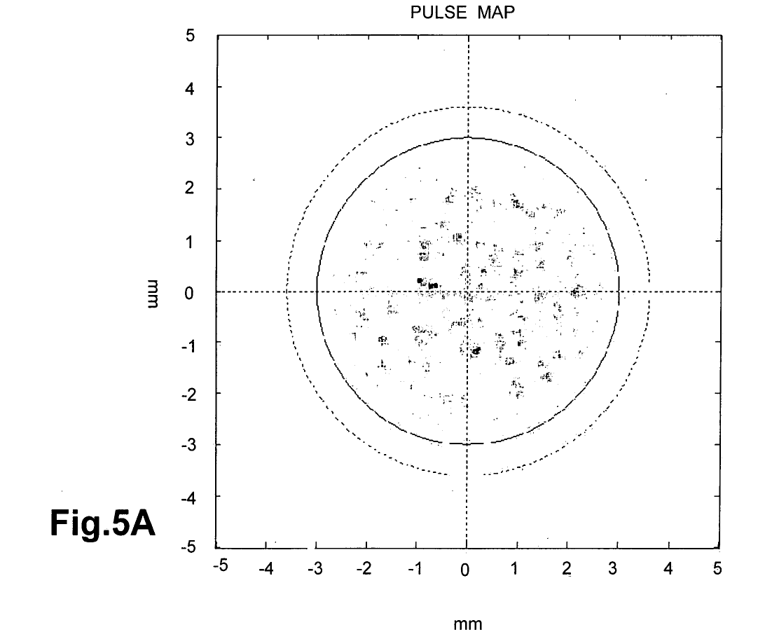

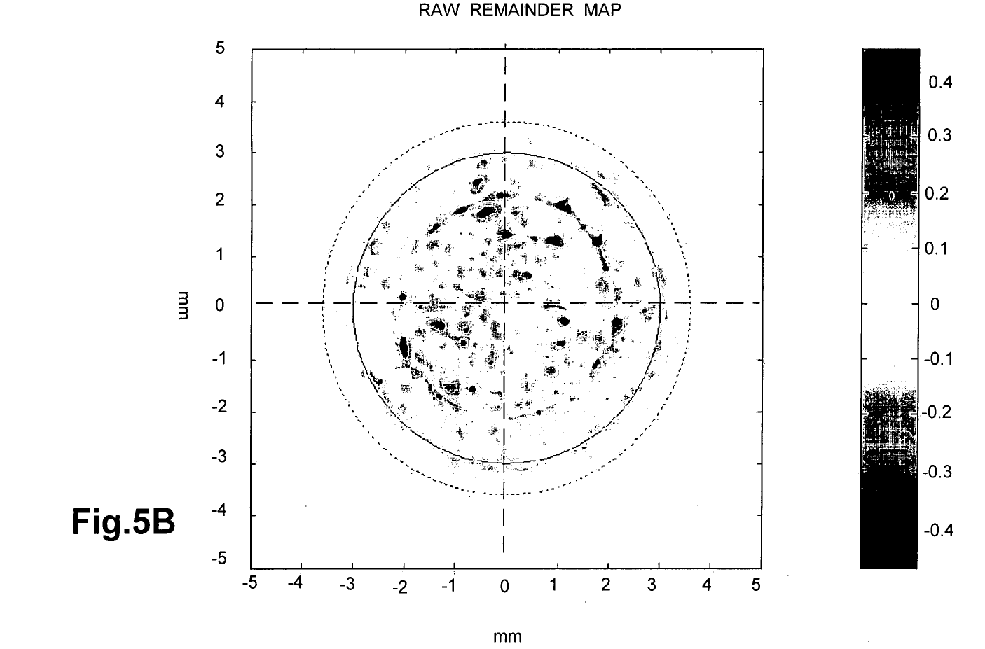

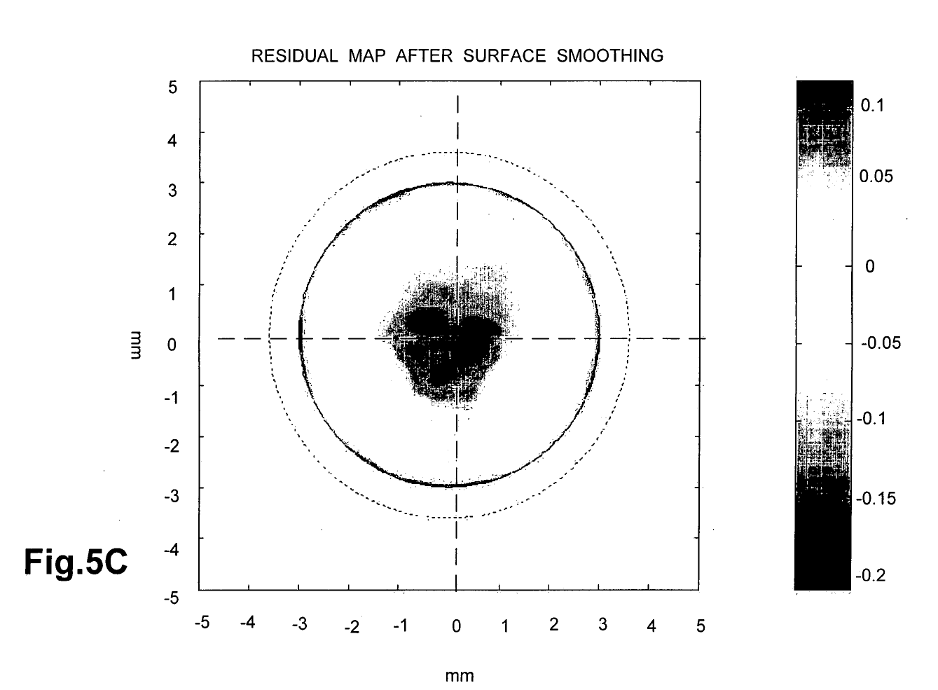

The pulse maps were generated for 1.0 D of myopia (FIG. 5A) and hyperopia. The rain-drop algorithm was able to use the larger 2-mm spot for more than half of the pulse counts and for the great majority of ablation volume (Table 2).

TABLE 2
The distribution of pulses generated by the rain-
drop algorithm using two different spots diameters. See FIGS. 4,
5 for corresponding ablation and pulse maps.

| | Spot | 2.0 | 1.0 | |
|---|---|---|---|---|
| | Diameter | mm | mm | Total |
| myopia | # spots | 524 | 456 | 980 |
| (−1.00 D) | % volume | 82.1 | 17.9 | 100 |
| hyperopia | # spots | 872 | 850 | 1722 |
| (+1.00 D) | % volume | 80.4 | 19.6 | 100 |

The remainder maps for the 1D ablations show variations on the scale of the 0.3 micron pulse ablation depth. The myopic remainder map is shown in FIG. 5B. The remainder map is the difference between the achieved ablation (pulse map convolved with the spot map) and the target ablation map a(x,y). The root-mean-square (RMS) values of the remainder maps for both the myopic and hyperopic examples are below the 0.3 micron pulse ablation depth (Table 3). After the expected surface smoothing, the achieved corrections deviate very little from the target corrections. The smoothed residual map for the myopic correction (FIG. 5C) shows a very slight under-correction pattern. The under-correction of 0.06 micron RMS is equivalent to −0.02D, which is clearly insignificant. The RMS smoothed residual for the hyperopic correction is similarly very small (Table 3). The smoothed residual map is defined as the difference between the achieved correction (pulse map convolved with the spot map and then smoothed according to Equation 14) and the target correction map (Equation 13).

TABLE 3
Root-mean-square (RMS) residual in simulated
ablations using 2 & 1 mm diameter spots.

| | | |
|---|---|---|
| myopia | Raw | 0.136 |
| (−1.00 D) | Smoothed | 0.058 |
| hyperopia | Raw | 0.131 |
| (+1.00 D) | Smoothed | 0.062 |

Sequential Non-Overlap Sorting Algorithm

It is also desirable to order the pulse sequences so that consecutive pulses are placed in non-overlapping locations. This prevents the plume of a laser pulse from affecting the transmission of subsequent pulses. The rain-drop algorithm already yields sequences with little sequential overlap. However, an additional sorting algorithm is preferably used to further minimize sequential overlap. This sorting operation is detailed in the following program code. First, the number of pulse intervals where the non-overlap condition must be observed is calculated by dividing the plume dispersal time by the laser firing interval and rounding up the result to the next larger integer. The pulses are examined consecutively in reverse order. The order was reversed because the clumping of pulses tends to occur late in the rain-drop algorithm. The pulse being examined is the index pulse. The spatial distance between the index pulse and the next pulse is compared with the required minimum separation (the spot diameter or slightly larger). If the separation is too small, the next pulse is exchanged with the nearest later pulse that satisfied the required separation with the index and previous pulses. The index is incremented until the end of the sequence is reached.

/* ALGORITHM TO SORT PULSE SEQUENCE FOR CONSECUTIVE NON-OVERLAP */
int i, j, k, m, t, flag, n_interval;
Location temp;
for (i = N_SPOT_SIZES−1; i >=0; i--) {

n_interval = ceiling(plume_dispersal_time[i] / PULSE_INTERVAL);
min_separation = PLUME_SPREAD * spot_diameter[i];
for (j = N[j]−1; j > 0; j--)

for (k = 0; k < n_interval; k++)

if (distance(sequence[i][j], sequence[i][j−k−1]) < min_separation)

for (m = j−k−2; m >= 0, m++){

flag = 1; /* If flag = 1, swappable*/
for (t = j−k, min(N(i), j−k−1+n_interval), t++)

if (distance(sequence [i][t], sequence

[i][m]) <= min_separation)

flag = 0;

if flag = 1{
temp = sequence[i][j−k−1];
sequence[i][j−k−1] = sequence[i][m];
sequence[i][m] = temp;
break;
}

}

}

/* END ALGORITHM */
/* COMMENTS
plume_dispersal_time[i] is the time needed to disperse the plume so it does not interfere with later pulses,
PLUME_SPREAD is the ratio between the effective plume diameter and the ablation spot diameter,
n_interval is the number of pulse interval that must elapse before overlap is allowed,
ceiling(number) returns the number rounded to the next larger integer, and
distance(pulse1, pulse2) returns the distance between pulse1 and pulse2.

*/

As an example, the sequence of the 2 mm spots in the 1 D myopia example are sorted. The non-overlap interval number was set to 1 (n_interval=1). The plume size was assumed to be the same as the ablation spot size (PLUME_SPREAD=1). The mean distance between consecutive pulses before sorting was 2.70 mm. Sorting improved this to 3.02 mm. The mean area overlap between consecutive pulses was 7.25% before sorting. This improved to 0.27% after sorting. The sorting algorithm is highly effective in reducing overlap. It has minimal effect on the fractal distribution of the pulse sequence because the swaps are few and random.

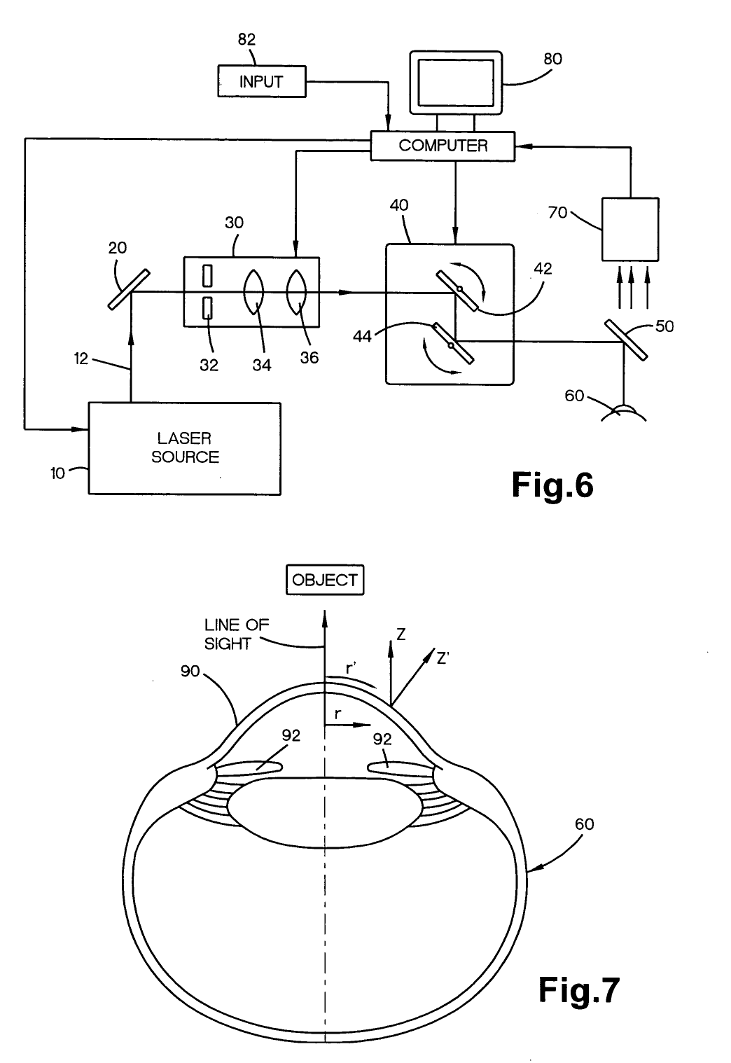

Referring to FIG. 6, a system is shown for practicing the present invention. A laser source 10 generates laser pulses that travels along beam path 12. These laser pulses are of the appropriate wavelength, duration, and energy for the intended corneal ablation. For example, an argon fluoride excimer laser operating at 193 nm can be used, with pulse duration in the millisecond range. The beam path is directed by mirror or mirrors 20 to beam shaping apparatus 30. Beam shaping is performed by a combination of an aperture 32, and lenses 34 and 36. The beam is then steered by scanning apparatus 40. The scanning apparatus preferably consists of two steering mirrors 42 and 44 along two perpendicular axes. The beam is then deflected by wavelength-selective mirror 50 onto the target eye 60. During the operation of the laser, the position of the eye is monitored by aiming apparatus 70. The aiming apparatus preferably consists of a microscope through which the surgeon can visualize the target eye, a video camera and other tracking sensors. The position of the eye can be measured by computer processing of the video images, or other types of tracking sensor output. Computer 80 is operatively connected to the beam shaping apparatus 30 and the scanning apparatus 40 for controlling those devices. The output of the arming-apparatus provides sensor information to computer 80. Computer 80 is operated by the laser operator through an appropriate input device 82 under the direction of the surgeon. The computer stores a pulse sequence and control algorithms described above which are used to control the laser pulse energy and timing, beam shaping, and beam steering. Input from aiming apparatus 70 is used to yield tracking information that modifies the instruction sent by computer 80 to steering apparatus 40.

An eye 60 is illustrated in FIG. 7. The cornea 90 is the target of the laser surgery used to correct the refractive error of the eye. The line of sight is the line connecting the center of the pupil with the object of regard. The pupil is the aperture formed by the iris 92. The Radius r of the global coordinate system is defined by the perpendicular distance (radius of the pupil) from the line of sight. The radius r′ of the local coordinate system is defined as the arc length from the line of sight along the anterior corneal surface. The z-axis of the global coordinate system is parallel to the line of sight. The z′-axis of the local cornice system is defined as being perpendicular to the corneal surface.

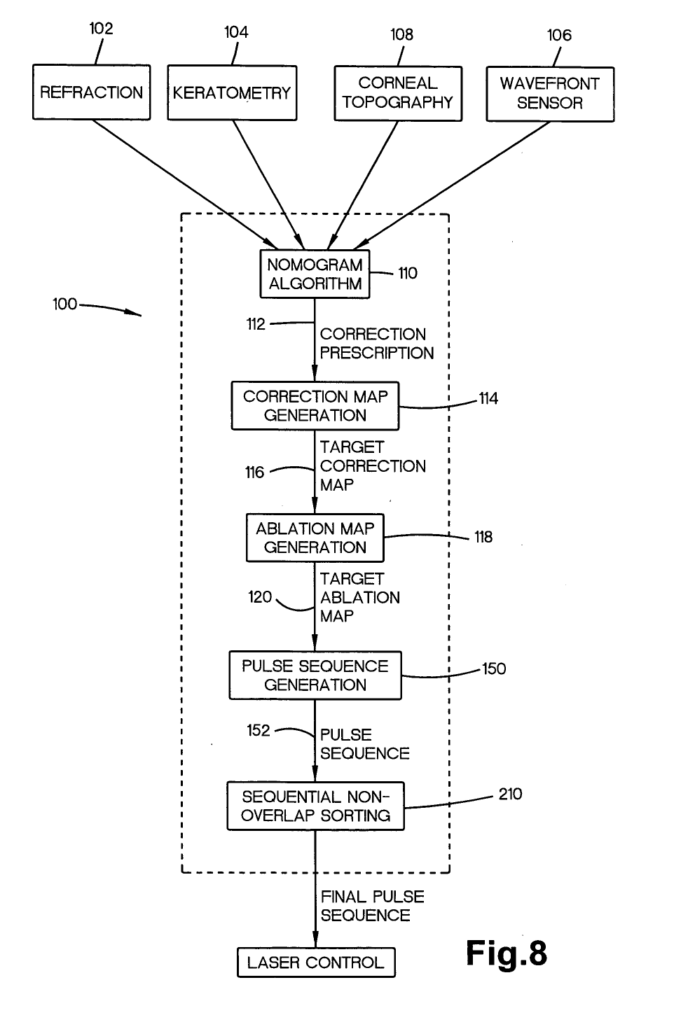

FIG. 8 is a top level flow chart of the ablation process 100 in accordance with the present invention. Conventionally clinical manifest refraction 102 is used to determine the setting of laser vision correction. Manifest refraction is determined by placing trial lenses in front of the subject eye and changing the lenses based on the subject's evaluation of the resulting vision, until the best-combination of lenses is found. This combination of spherical and cylindrical lenses is termed the manifest refraction prescription. Manifest refraction can be performed after the eye ciliary muscle has been paralyzed with eye drops. This is termed “cycloplegic refraction”. The surgeon may also modify the laser setting using keratometry 104, which measures the corneal curvature at one single diameter. Wavefront sensors 106 can be used to measure the detailed optical aberration of the eye, including spherocylindrical refraction and higher order aberration. The output of the wavefront sensor 106 is a map of wavefront height, which can be further analyzed to provide the amplitude coefficients for Zernike series. Corneal topography 108 provides a map of the anterior corneal surface height. This provides more detailed information than keratometry and can also be analyzed to provide Zernike coefficients. All this measured and derived data can be combined to provide a prescription for laser correction. If the prescription only consists of spherocylindrical terms (or the equivelant Zernike 2^{nd} order terms) then it is a traditional refractive prescription. If it contains higher order Zernike terms, this is a wavefront prescription. All prescription information is input to a nomogram algorithm 110 which is a correcting algorithm used to calculate actual settings for a laser system to achieve correction. The output 112 of the nomogram algorithm is referred to as the correction prescription. The correction prescription 112 is the input for the correction map-generating algorithm 114 of the present invention. The resulting output 116 is the target correction map, which is specified in terms of the desired corneal surface height change. The target correction map output 116 is input to an ablation map-generating algorithm 118 that converts this to a target ablation map 120. The target ablation map 120 specifies the depth of laser ablation to be performed.

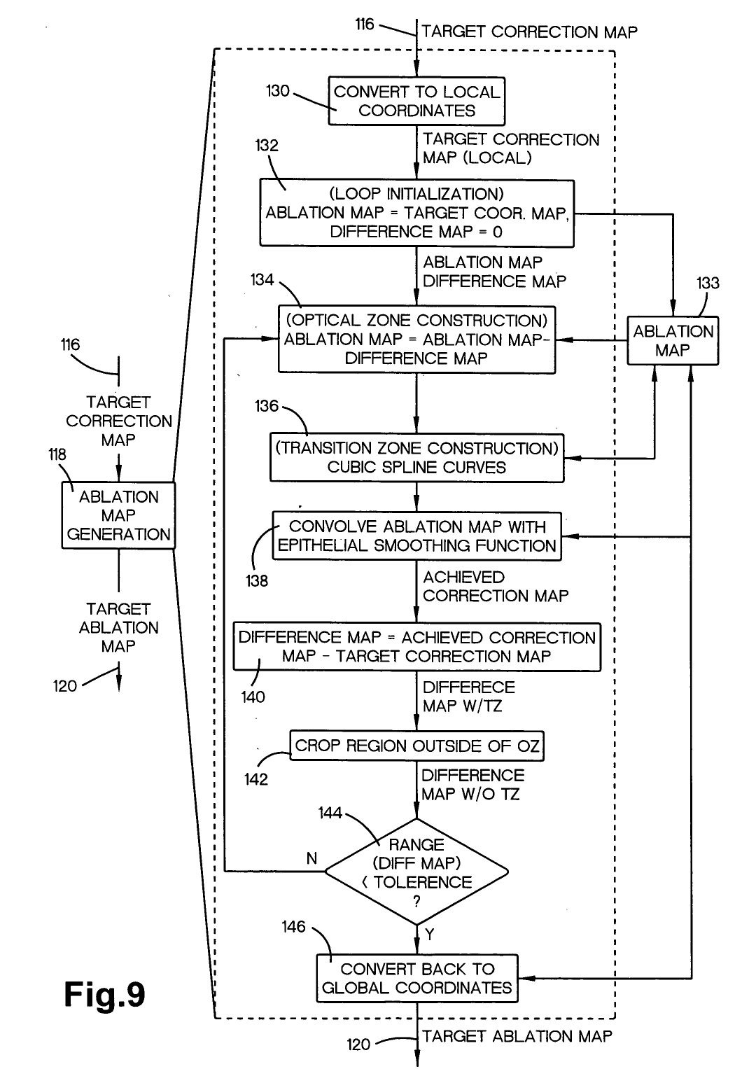

Referring to FIG. 9, details of the ablation map generating step 118 are shown in detail. The target correction map output 116 is provided to a conversion step 130 that converts the map to local coordinates and provides the target correction map in local coordinates to a loop initialization step 132. In step 132, the ablation map value is set equal to the target correction map and the difference map value is set equal to zero. The ablation map 133 is the stored ablation map that is being operated on by the subroutine at the various steps. Those skilled in the art will appreciate that the ablation map is called up from storage, operated on in accordance with a process step and then returned to the same storage memory. In step 134, optical zone construction occurs in which the ablation map value is set equal to the ablation map value minus the difference map value. This provides an ablation map without a transition zone. In step 136, the transition zone is constructed using cubic spline curves discussed above which provide smoothing. Next, a convolve ablation map with epithelial smoothing function is performed in step 138 to derive an achieved correction map. The difference map value is then set equal to the achieved correction map minus the target correction map in step 140 that is a difference map with a transition zone. The region outside of the optical zone is cropped in step 142 to yield a difference map without a transition zone. In step 144, a determination is made as to whether the difference map range is less than a predetermined tolerance value. If the determination is negative, the process loops back to step 134. If the determination is affirmative, the process then-converts the map back to global coordinates in step 146 and outputs the target ablation map 120.

Referring back to FIG. 8, the target ablation map 120 is input to a pulse sequence generating step 150 that converts the target ablation map into a pulse sequence 152. The pulse sequence 152 specifies the location, diameter, and fluence profile of laser pulses. In addition, the pulse sequence contains order, size, and locations of the laser pulses.

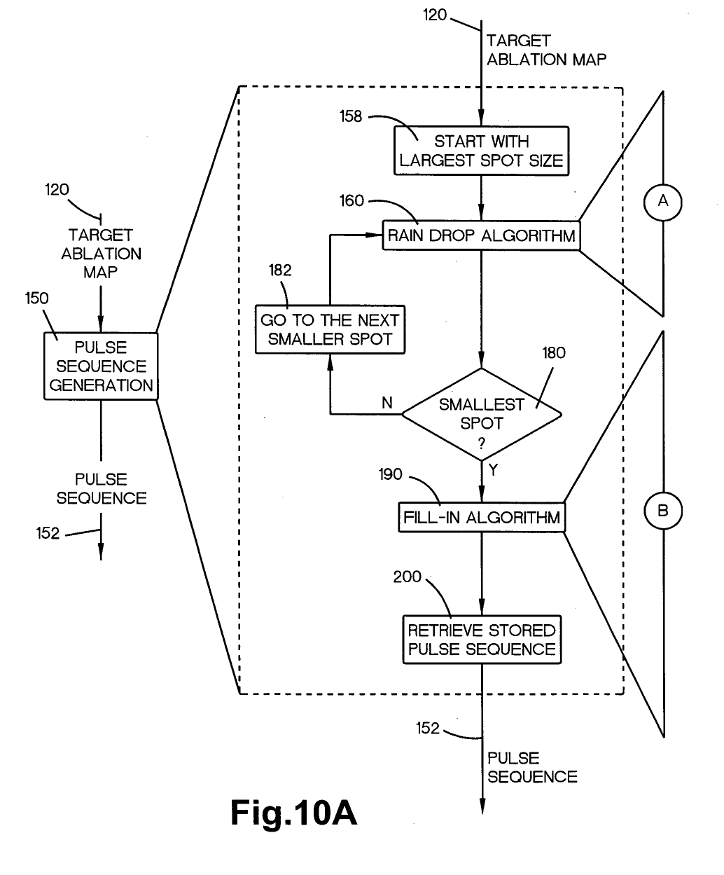

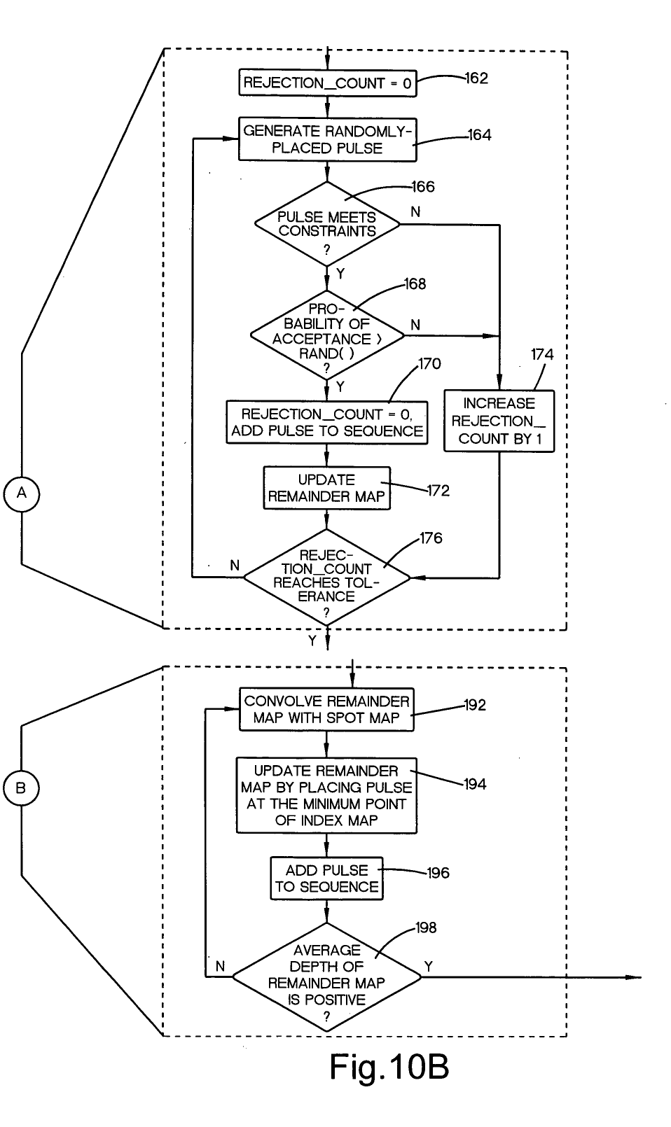

Referring to FIG. 10, details of the pulse sequence generation step 150 is shown. As discussed above, the largest spot size of the target ablation map 120 is first analyzed using the rain-drop algorithm 160. In the rain-drop algorithm, the rejection count value is set equal to zero in step 162. Randomly placed pulses are then generated in step 164. A determination is made in step 166 as to whether the pulse meets constrains. The constraints are (1) Ablation Zone—the pulse fits within the ablation zone, (2) Non-Coincidence—the pulse does not coincide with other pulses, and (3) Non-Positivity—the pulse does not cause any point on the remainder map to become positive. Recall, only negative values are permitted. If the determination in step 166 is affirmative, a determination is made in step 168 as to whether the probability of acceptance is greater than random. The probability of acceptance is the average depth of the remainder map inside the pulse spot divided by the deepest depth on the entire remainder map. The random number is chosen from a uniform distribution on the interval between zero and one.

If the determination is step 168 is affirmative, the process sets the rejection count to zero and adds the pulse value to the sequence in step 170 and the remainder map is updated in step 172. The remainder map is the difference between the achieved ablation and the target ablation map. If either determination 166 or 168 is negative, the process increases the rejection count by 1 in step 174. From either step 172 or step 174, a determination is made in step 176 as to whether the rejection count has reached a predetermine tolerance. If the determination in step 176 is negative, the process loops back to step 164. If the determination in step 176 is affirmative, a determination is made in step 180 as to whether the smallest spot has been processed. If the determination is negative, the next smallest spot is determined in step 182 and the rain-drop algorithm is run on the next smallest spot.

If the determination in step 182 is affirmative, the fill-in algorithm is run in step 190. In the fill-in algorithm 190, a convolved remainder map with spot map is provided in step 192. The remainder map is then updated in step 194 by placing the pulse at the minimum point on the index map. A pulse is then added to the sequence in step 196. A determination is then made in step 198 as to whether the average depth of the remainder map is positive. If the determination is negative, the process loops back to step 192. If the determination in step 198 is affirmative, the process then retrieves the stored pulse sequence in step 200 and the pulse sequence 152 is then provided.

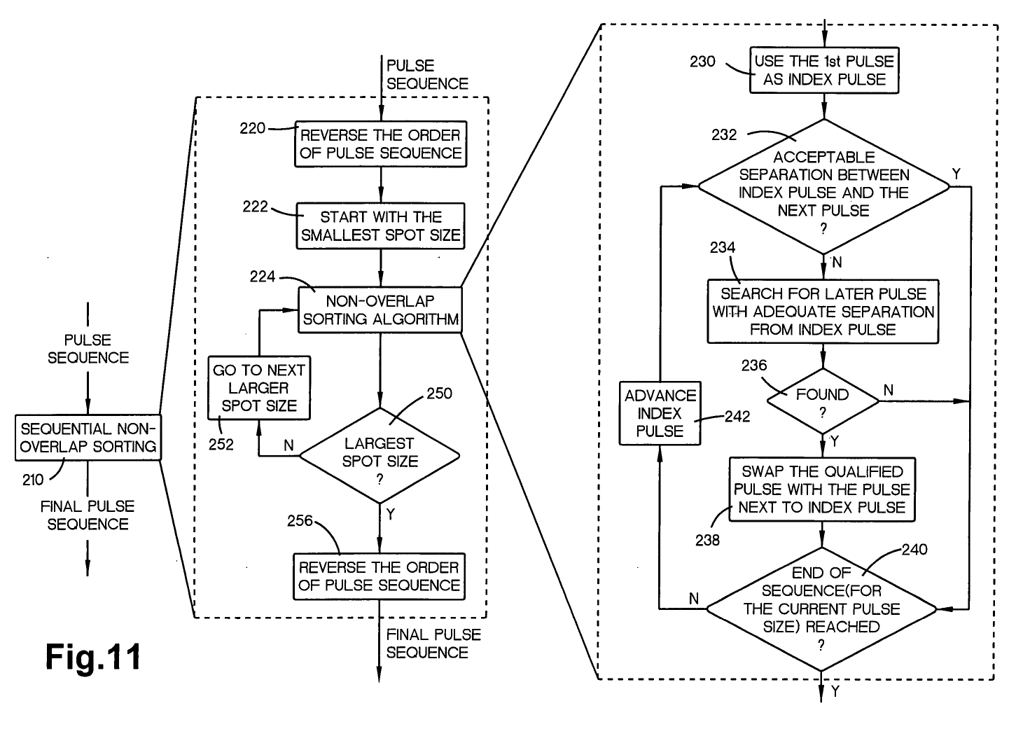

Referring back to FIG. 8, the pulse sequence 152 is provided to the sequential non-overlap sorting step 210. Referring to FIG. 11, the details of the sequential non-overlap sorting step 210 will be appreciated. The order of the pulse sequence is reversed in step 220 and the smallest spot is identified in step 222 for further processing. In step 224, the non-overlap sorting algorithm is performed. In this sorting algorithm, the first pulse is used set as the index pulse in step 230. In step 232, a determination is made as to whether there is an acceptable separation between the index pulse and the next pulse. If the determination in step 232 is negative, the process proceeds to step 234 where a later pulse with adequate separation from the index pulse is identified. A determination is then made in step 236 as to whether such a later pulse, i.e., the pulse searched for in step 234, has been found. If the determination in step 236 is affirmative, the process then swaps the qualified pulse with the pulse next to the index pulse in step 238. From either an affirmative determination is step 232, a negative determination in step 236, or from step 238, the process makes a determination in step 240 as to whether the end of the sequence for the current pulse size has been reached. If the determination is step 240 is negative, the index pulse is advanced in step 242 and the process then loops back to step 232.

If the determination in step 240 is affirmative, the process makes a determination in step 250 as to whether the spot size processed was the largest spot size. If the determination in step 250 is negative, the process goes to the next large spot size in step 252 and then loops back to step 224. If the determination in step 250 is affirmative, the process then reverses the order of the pulse sequence in step 256 and outputs the final pulse sequence. Referring back to FIG. 8, the final pulse sequence is then provided to the laser control for vision correction.

Construction of Mathematical Model to Account for Corneal Surface Smoothing after Laser Refractive Surgery

A model of epithelial transport is proposed based on first principles. The first postulate is that epithelium migrates toward a depression on the corneal surface. Mathematically, this is similar to the flow of solute across a concentration gradient and can be described by the partial differential equation below. For a corneal surface region S bound by a closed line L, the rate at which epithelial volume flows out of a line element dl depends on the surface height gradient.
−dQₘ−μ(∇h′)·n dl
where
Qₘ is the migratory epithelial volume flow,
μ is the motility of epithelium,
∇ is the gradient vector operator in two dimensions i ∂/∂x′+j ∂/∂y′, where x′ and y′ are local transverse coordinates tangential to the surface,
h′ is the corneal surface height along the z′ axis, which is defined locally as normal to the surface, and
n is the outward vector normal to the line element dl.

The net migratory flow of epithelial volume into the area enclosed by boundary L is given by a line integral.
Qₘ=∫_{L}μ(∇h′)·n dl Equation 33

Application of the divergence theorem in two dimensions gives us a surface integral.
Qₘ=∫∫ₛμ(∇²h′)dσ Equation 34
where
∇² is the Laplacian operator in two dimensions ∂²/∂x′²+∂²/∂y′², and dσ is the surface element.

Epithelial cells divide and provide replacement to the epithelial volume. It is further postulated that cell division occurs at a constant rate. Although this may not be true in an active wound healing phase, it should be close to reality in the state of equilibrium. In integral form, this event can be expressed as:
Q_{g}=∫∫ₛγdσ Equation 35
where
Q_{g} is the generative epithelial volume flow, and
γ is the generativity of epithelium (rate of epithelial growth or replacement).

Epithelial cells mature and move to the surface, where they eventually slough off. It is postulated that the rate of loss is proportional to the thickness of the epithelium p′.
Q₁=∫∫ₛ−λp′dσ Equation 36
where
Q₁ is the epithelial volume flow due to loss,
λ is the lossivity of epithelium (rate of epithelial sloughing), and
p′ is the epithelial thickness measured along the surface normal axis z′.

To complete the picture of epithelial transport, the epithelial thickness is linked to corneal surface height.
h′=s′+p′ Equation 37
Qₑ=∫∫ₛ∂p′/∂tdσ Equation 38
where
s′ is the subepithelial surface elevation, and
Qₑ is the absorption of epithelial volume flow by epithelial thickness change.

Conservation of epithelium provides the master equation.
Qₑ=Qₘ+Q_{g}+Q₁ Equation 39

In integral form, the equation expands to:
∫∫ₛ∂p′/∂t−μ(∇_{L}²h′)−γ+λp′dσ=0 Equation 40
Since the model applies to any region S on the corneal surface, the equation can be written in the differential form where the integrand vanishes.
∂p′/∂t−μ(∇²h′)−γ+λp′=0 Equation 41

In equilibrium, ∂p′/∂t=0 and therefore
−μ(∇²h′)−γ+λp′=0 Equation 42

The equation is simplified to obtain measurable constants.
p′=c+s²∇²h′, Equation 43
where
c=γ/λ represents the constant component of the epithelial thickness determined by a balance between growth and loss. The constant c has a unit of length (thickness).
s=√(μ/λ) is the smoothing constant. It has a unit of length and can be thought of as the radius over which smoothing occurs. It is determined by a balance between epithelial migration and loss. One can also think of it as the typical distance over which epithelium migrates before it sloughs off. The term s ∇²h′ describes the variable component of the epithelial thickness that responds to surface curvature changes.

The above model is applied to the prediction of corneal surface smoothing after LASIK and PRK. Equation 44 below states that the post-operative corneal surface height h₁(x′,y′) is equal to the pre-operative height h′₀(x′,y′) plus the ablation a′ (x′,y′) (depth as negative quantity) and the change in epithelial thickness Δp′ (x′,y′).
h′₁=h′₀+a′+Δp′ Equation 44
where Δp′=p₁′−p₀′

Using Equation 43, we find that the change in epithelial thickness is:
Δp′=s²∇²Δh′ Equation 45
where
Δh′=h₁′−h₀′

Applying Equation 45 to Equations 44, we find
Δh′=a′+s²∇²Δh′ Equation 46

Equation 46 has solutions in the forms of separable complex exponential functions exp(jωₓ′x′)=cos(ωₓ′x′)+j sin(ωₓ′x) and exp(jω_{y}′y′)=cos(ω_{y}′y′)+j sin(ω_{y}′y′). Therefore, it is useful to look at it in the frequency domain. Equation 47 is derived from Equation 46 using the differential property of the Fourier Transform.
ΔH′=A′+s²(ωₓ′²+ω_{y}′²)ΔH′ Equation 47
where
ΔH′ (ωₓ′, ω_{y}′) is the 2-dimensional Fourier Transform of Δh′(x′,y′),
A′ (ωₓ′, ω_{y}′) is the 2-dimensional Fourier Transform of a′ (x′,y′), and
ωₓ′ and ω_{y}′ are the respective spatial radian frequencies for x′ and y′ in radians/length.

Equation 47 can be expressed using a transfer function F′ (ωₓ′, ω_{y}′).
ΔH′=F′A′ Equation 48A
F′(ωₓ′,ω_{y}′)=1/[1+(ωₓ′/ω_{c}′)²+(ω_{y}′/ω_{c}′)²] Equation 48B
where the cutoff radian frequency is ω_{c}′=1/s in radian/length.

The transfer function F′ is identical to the square of a first-order Butterworth low-pass filter in two dimensions. Thus, the action of the epithelium is theoretically identical to a low-pass filter. In other words, the low-pass filter F′ characterizes the frequency response of the corneal surface.

The local coordinate system x′, y′, z′ has been used. The local coordinate system has the perspective of a small-epithelial cell on the corneal surface and is important to the development of the epithelial smoothing theory. However, the corneal height and ablation depth are usually specified in fixed global coordinate system x, y, z. In the global coordinate system, z is defined as the line of sight and x, y are perpendicular to z. The global coordinate system has the perspective of the laser scanning system looking down at the cornea. The coordinate systems are identical at the corneal apex but the deviation increases as the radial distance approaches the corneal radius of curvature.

The following approximate transform equations are used to convert between the local and global coordinates. The transform treats the corneal surface as a perturbation from a sphere. These are approximations given that the epithelial smoothing change is a small correction on the effect of the ablation and the transform is a secondary correction on the epithelial smoothing calculation.
a′(r′,θ)=a(r,θ)cos[arcsin(r/R_{C})] Equation 49A
r′=R_{C} arcsin(r/R_{C}) Equation 49B
Δh(r,θ)=Δh′(r′,θ)/cos(r′/R_{C}) Equation 49C
r=R_{C} sin(r′/R_{C}) Equation 49D
where
r is the radius perpendicular to the central axis defined by the line of sight,
R_{C} is the radius of curvature of the corneal best-fit sphere (a value of 7.6 mm is used in our simulations),
height h is measured along the z axis, which is positive outward along the line of sight,
x is perpendicular to the line of sight and positive to the right of a viewer facing the eye,
y is perpendicular to the line of sight and positive upward,
θ is the meridian angle, which is zero on the +x axis and increases counterclockwise, and
r′ is the arc length from the corneal apex.

The coordinate transforms in Equation 49 are performed in polar coordinates and the low-pass filter in Equation 48 is specified in Cartesian coordinates. To convert between the polar and Cartesian coordinate systems, the following equations are used.
x=r cos θ Equation 50A
y=r sin θ Equation 50B
r=\sqrt{(x²+y²)} Equation 50C
θ=arctan(y/x) Equation 50D
x′=r′ cos θ Equation 51A
y′=r′ sin θ Equation 51B
r′=\sqrt{(x′²+y′²)} Equation 51C
θ=arctan(y′/x′) Equation 51D

Ablation simulations can be performed using MatLab software (The Mathworks, Inc. Natick, Mass., USA). The corneal surface is simulated as a sphere of 7.6 mm radius. Surfaces, ablations, and surface changes are computed on digital grids of 10×10 mm with a sampling interval of 0.02 mm. The ablation maps are based on modifications (see specifics below) of the exact Munnerlyn's algorithm for target 1 diopter (D) corrections. The ablation map is Fourier transformed to the frequency domain. The smoothing action is simulated with the low-pass filter in Equation 48 using smoothing constants ranging between 0 and 1 mm. Then the corneal surface height change in converted from the frequency domain to the spatial domain. The smoothed surface change is applied to the simulated corneal surface.

The surgically-induced refractive change is evaluated using Zernike polynomial decomposition Zernike polynomial decomposition is applied to the corneal surface height to obtain coefficients h_{n,m}, which are the amplitudes of the Zernike terms Zₙ^{m}. The change coefficients Δh_{n,m} are computed by taking the difference between post-operative and pre-operative coefficients. The achieved refractive effect of the corneal surface change is evaluated with the formulae given by Schwiegerling et al. (J Opt. Soc. Am. A, 1995, “Representation of Videokeratosdocopic Height Data With Zernike Polynomials” 12 (10):p. 2105-2113) with the substitution of corneal refractive index in place of keratometric index. The change in the coefficient Δh_{2,0} of the defocus term Z₂⁰ is used to evaluate spherical equivalent (SE) change (Equation 52A). The change in the coefficients Δh_{2,±2} of the astigmatism terms Z₂^{±2} is used to evaluate astigmatism correction (Equation 52B). A 5.0 mm pupil diameter is used for the Zernike analysis of refraction because it is the average pupil size in the dim lighting from a projected eye chart. Five mm is also in between the average pupil sizes in darkness (5.8-6.1 mm) and in room light (4.1 mm). The correction/ablation ratio is the achieved dioptric correction for the 1D target ablation. The ratio is normalized to 1 for s=0.

$$\begin{matrix}Δ SE = ( n - 1 ) \frac{4 \sqrt{3}}{{R}_{P}^{2}} Δ {h}_{2 , 0} & Equation 52 A \\ Δ Astigmatism = ( n - 1 ) \frac{4 \sqrt{6}}{{R}_{P}^{2}} \sqrt{{( Δ {h}_{2 , 2} )}^{2} + {( Δ {h}_{2 , - 2} )}^{2}} & Equation 52 B\end{matrix}$$

where
ΔSE=change in spherical equivalent in dioptric unit,
Δastigmatism=magnitude of astigmatism change vector in dioptric unit,
Rₚ=pupil radius in mm, a value of 5.0 is used in our calculations,
n=corneal refractive index., a value of 1.377 is used,
Δh_{2,0}=change in coefficient for Z₂⁰ in μm, and
Δh_{2,±2}=change in coefficient for Z₂^{±2} in μm.

Spherical aberration is assessed with the coefficient Δh_{4,0} of the Zernike term Z₄⁰. This was evaluated using both 5 and 6 mm diameter analytic zones (pupils).

Simulation of Hyperopic and Myopic Ablations with a LADARVision

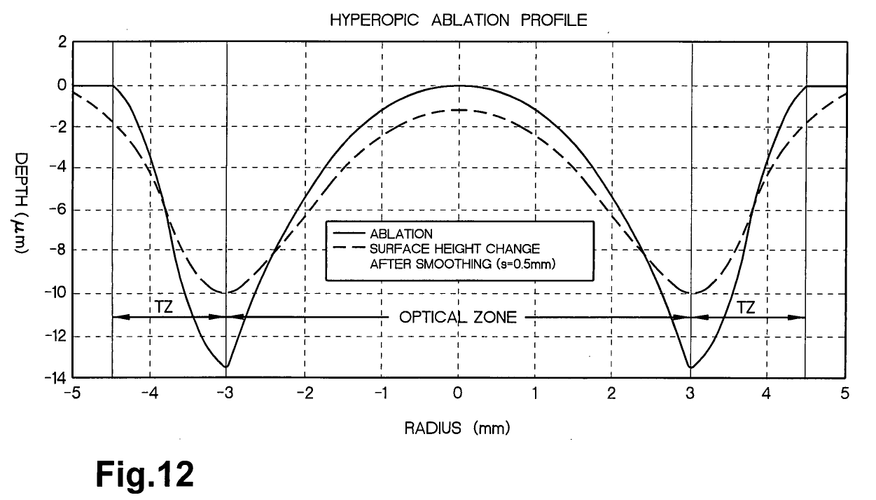

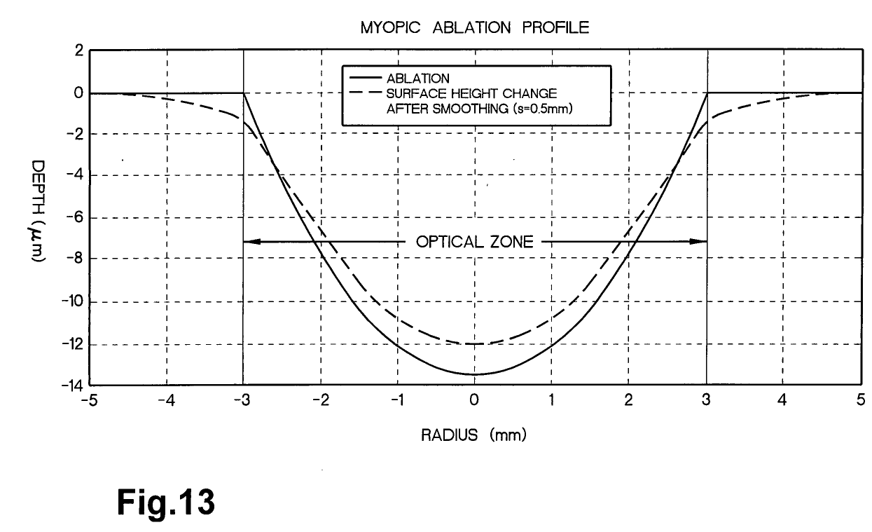

Simulations of myopic (−1D) and hyperopic (+1D) ablations were performed for comparison to the observed degrees of under-corrections with the LADARVision laser system (Alcon Summit Autonomous, Orlando, Fla.). The hyperopic ablation has a 6.0 mm diameter optical zone (OZ) and 9.0 mm transition zone (TZ). The exact Munnerlyn algorithm is used in the OZ. A cubic spline transition starts and ends with slope of zero in the TZ (FIG. 12). The myopic ablation has a 6.0 mm diameter with no TZ (FIG. 13).

Simulation of Minus Cylinder Ablation With a EC-5000

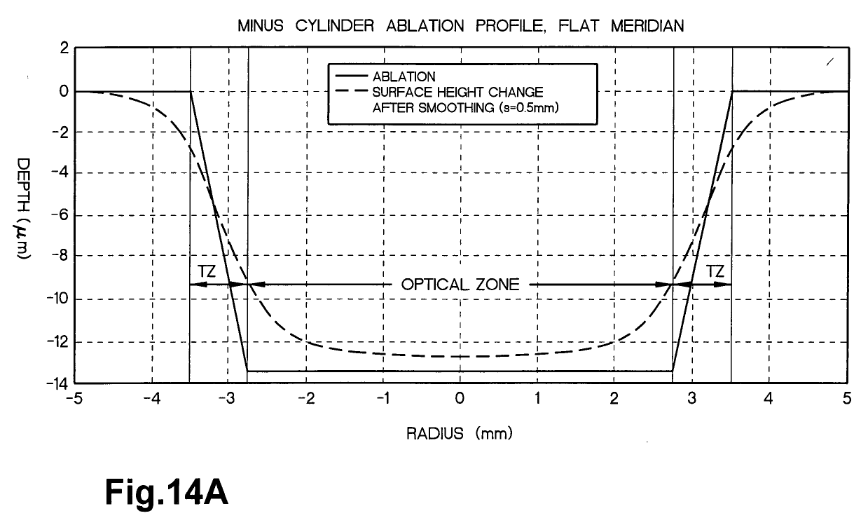

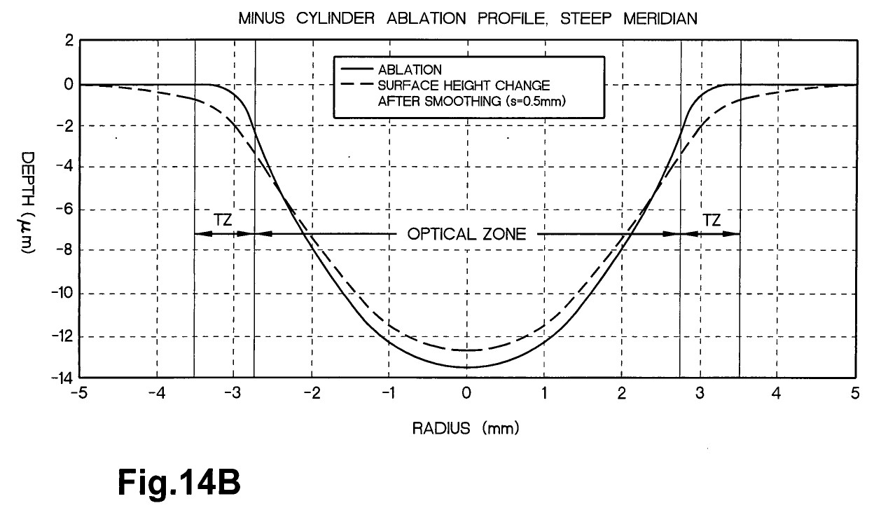

Simulation of 1 D of minus cylinder ablation on the EC-5000 laser (Nidek, Japan) was performed for comparison to published data. The EC-5000 ablation algorithm utilizes a circular OZ of 5.5 mm with a transition zone (TZ) out to 7.0 mm diameter. The laser ablation is shaped by both an expanding-slit and an expanding-circular aperture. The action of the slit aperture was simulated by a full Munnerlyn-algorithm cylindrical correction in the OZ with continuous tapering in the TZ. The circular aperture provides a constant-slope transition in the TZ. The ablation map is the product of the two aperture effects. The ablation profiles in the flat and steep meridians are shown in FIGS. 14A and 14B, respectively.

Clinical data analysis was based on a computer database for laser in situ keratomileusis (LASIK). Cases were reviewed over, approximately a 18 month period for inclusion in the regression analysis. The inclusion criteria were:
1. Spherical hyperopic or myopic corrections.
2. No previous eye surgery.
3. No astigmatism correction with the laser (refractive astigmatism<0.5 D).

4. Pre- and post-operative best corrected visual acuity of 20/20 or better.

5. Optical zone of 6.0 mm on laser setting.

6. No flap complication.

The laser settings were based on manifest and cycloplegic refractions and corneal topography (C-Scan, Technomed, Germany). The Hansatome (Bausch & Lomb Surgical, St. Louis, Mo.) was used to create superiorly hinged flaps. Laser ablation was performed using the LADARVision laser system (Alcon Summit Autonomous, Inc., Orlando, Fla.). Refraction obtained at the three month post-operative visit was used for outcome analysis. The database was maintained using spread sheet software. Statistical analysis was performed using JMP software Version 4 (SAS Institute, Cary, N.C.). Surgically-induce refractive change (SIRC) was calculated by taking the difference between post-operative and pre-operative manifest refractions. The spherical equivalent (SE) component of refraction was calculated by adding the spherical component to half of the cylinder magnitude.

Results

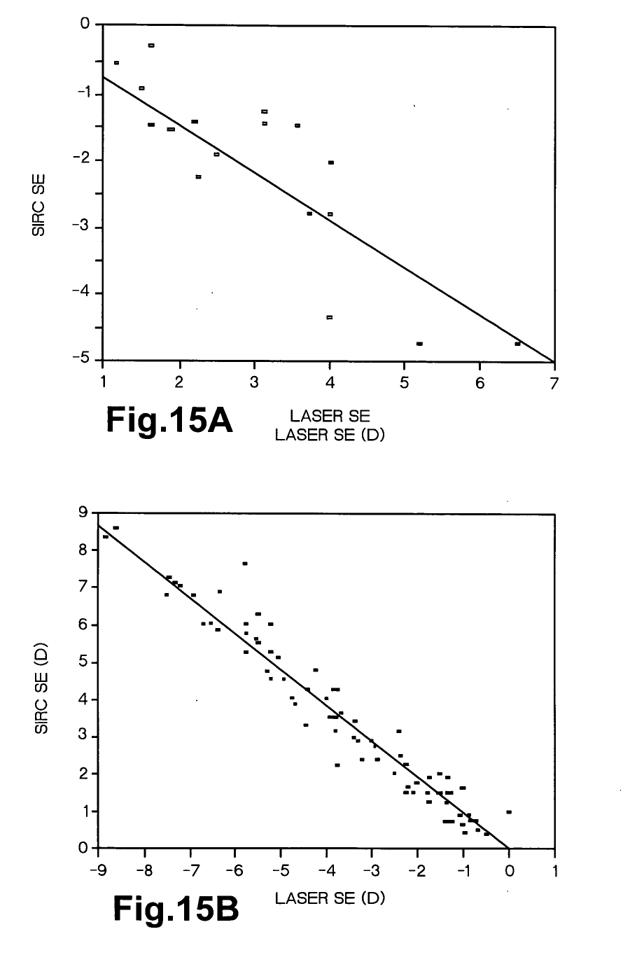

Refractive outcome for 19 cases of spherical hyperopic LASIK and 77 cases of spherical myopic LASIK were analyzed. All cases utilized a 6 mm diameter OZ. Linear regression showed that each diopter of hyperopic ablation produced 0.708 D of effect (FIG. 15A). The laser setting analyzed was the actual ablation setting that included laser's internal nomogram adjustment, which adds 50% ablation to entered hyperopic settings up to 2 D and adds 1 D for entries of more than 2 D. Using the same laser, each diopter of myopic ablation produced 0.968 D of correction (FIG. 15B). The clinical correction/ablation ratios of hyperopic and myopic ablations are significantly different (p.<0.001). Since the series are produced at the same center, with the same surgeon, laser, optical zone diameter, and surgical technique, it is clear that hyperopic ablation is subject to more regression or other modifying effects than myopic ablation.

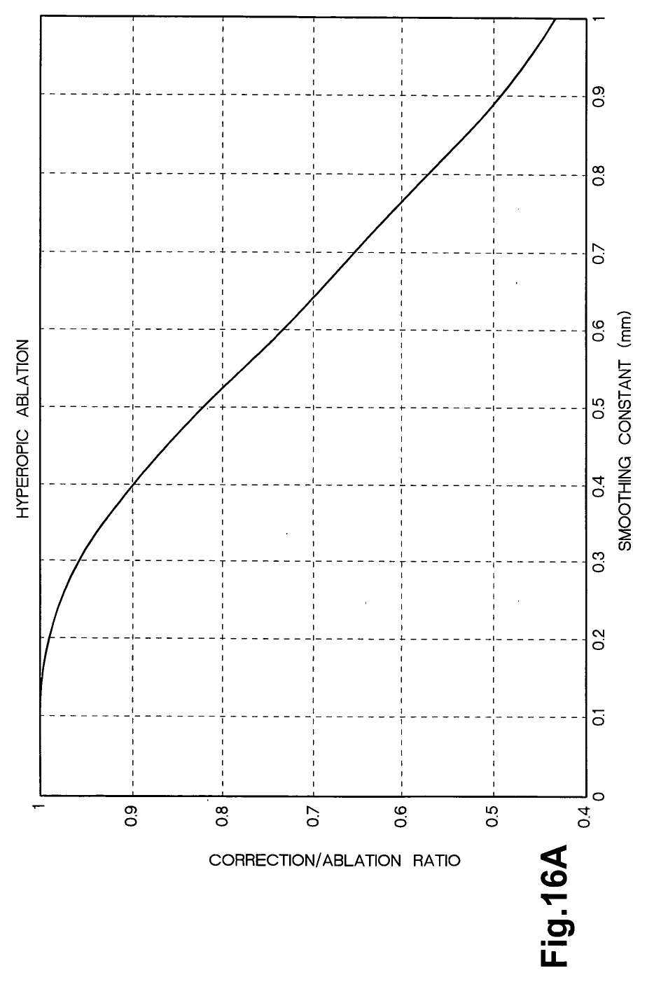

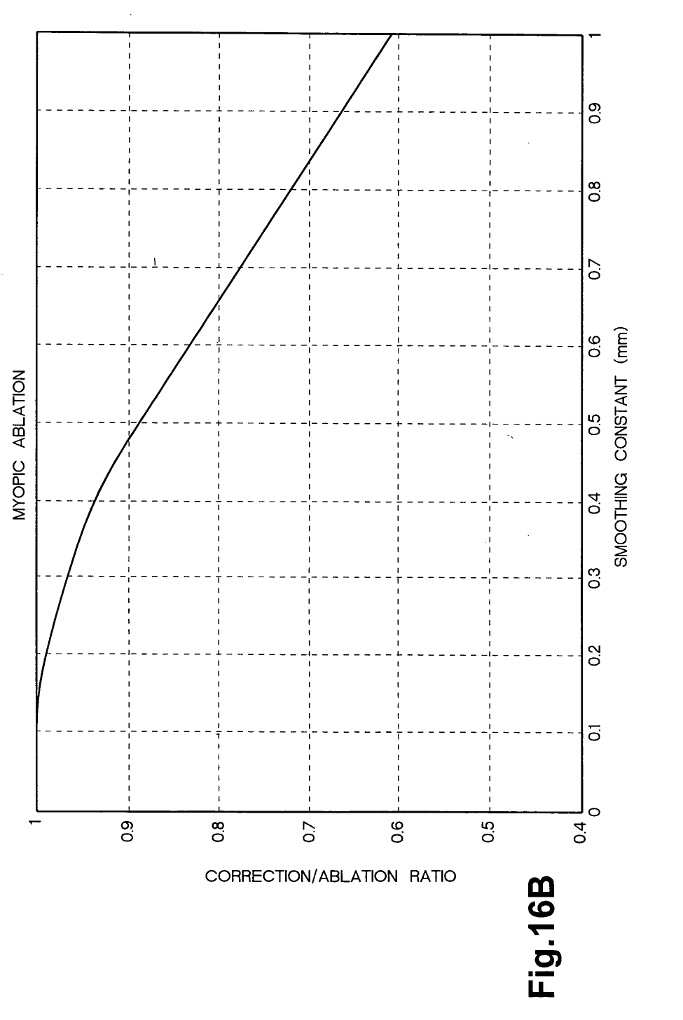

The simulations showed that the correction/ablation ratios for both-hyperopic and myopic ablations decrease with increasing smoothing action, which is controlled in the model, in accordance with the present invention, by the smoothing constant S. With increasing s, the correction/ablation ratio fell faster for hyperopia (FIG. 16A) than for myopia (FIG. 16B). But the difference was not as great as the clinical data indicated (FIG. 4). A correction/ablation ratio of 0.708 for hyperopia correction is matched at s=0.63 mm. A correction/ablation ratio of 0.968 for myopia correction is matched at s=0.32 mm.

The reduced correction is correlated with epithelial thickness modulation. In FIGS. 12 and 13, the epithelial thickness changes can be visualized as the difference between the achieved corneal surface change profiles and the ablation profiles. After hyperopic ablation (FIG. 12), the model predicts epithelium thinning at the center and thickening at the periphery of the OZ, where the ablation is deepest. After myopic ablation (FIG. 13), the model predicts epithelium thickening at the center and thinning at the periphery of the OZ.

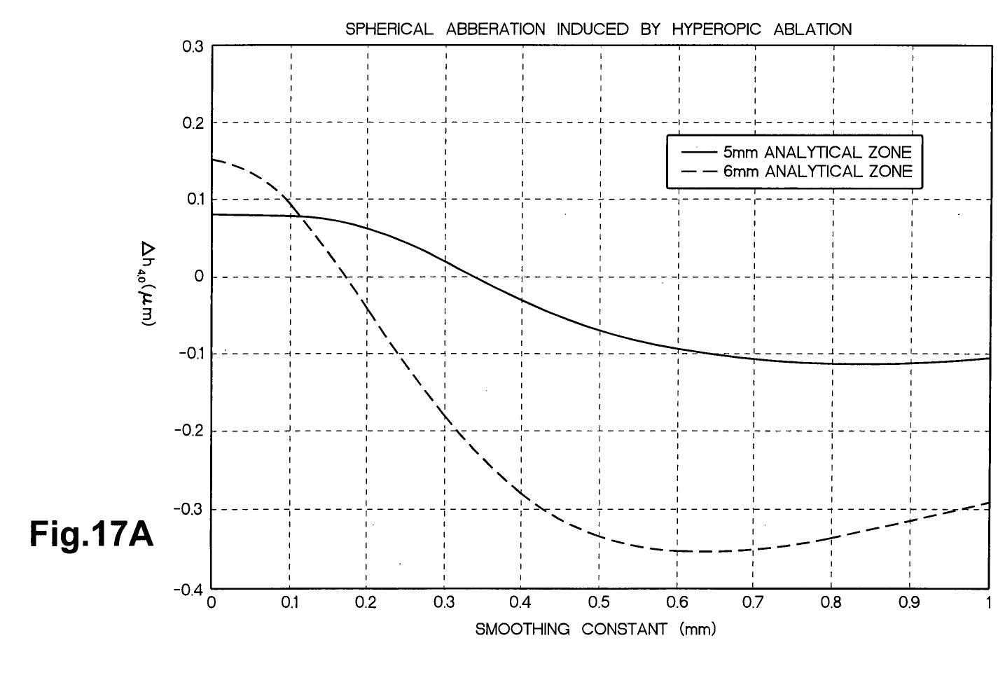

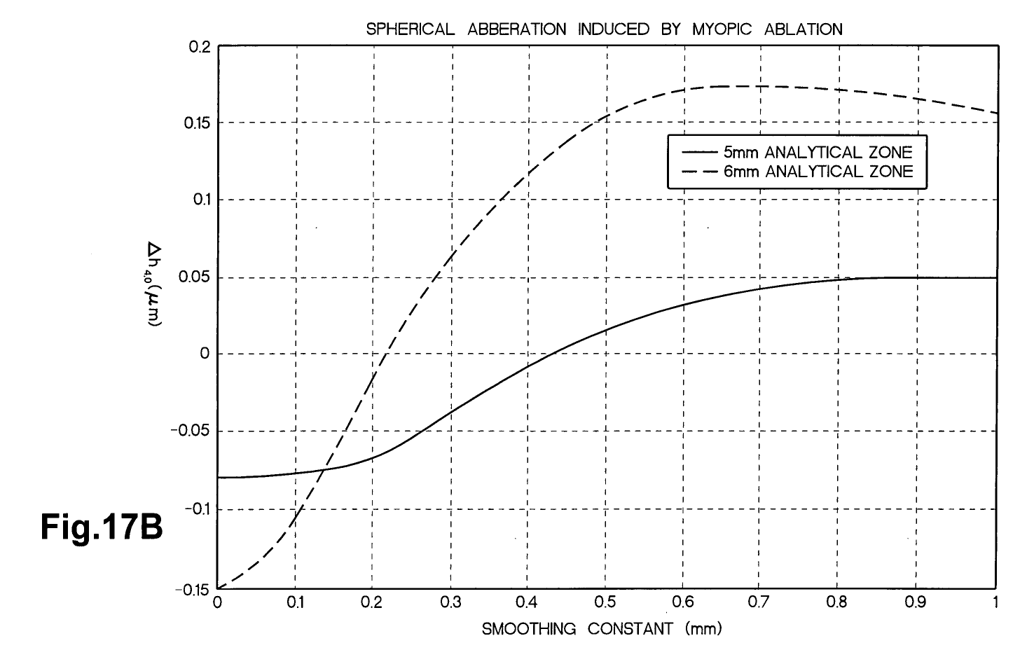

The simulations show that, in the absence of smoothing (s=0), there is induction of oblate spherical aberration (SA) after hyperopic ablation (FIG. 17A) and prolate SA after myopic ablation (FIG. 17B). This is expected from the spherical nature of Munnerlyn ablation algorithms. However, with increased smoothing, the opposite SA is induced. At s 0.5 mm, prolate SA is induced after hyperopic ablation (FIG. 17A) and oblate SA is induced after myopic ablation (FIG. 17B).

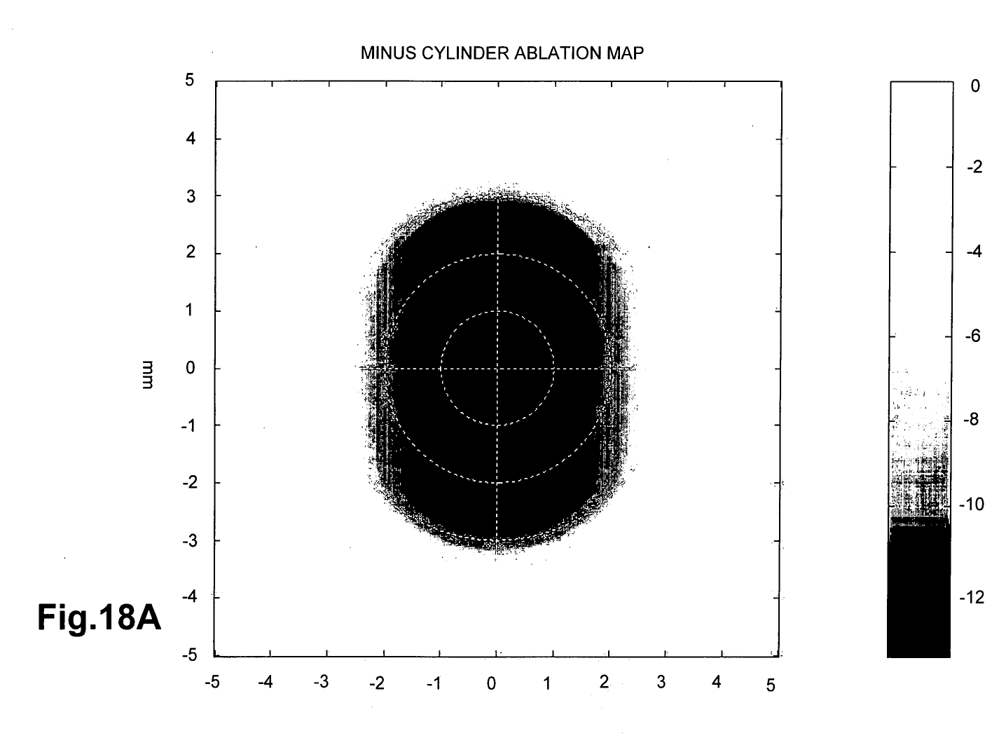

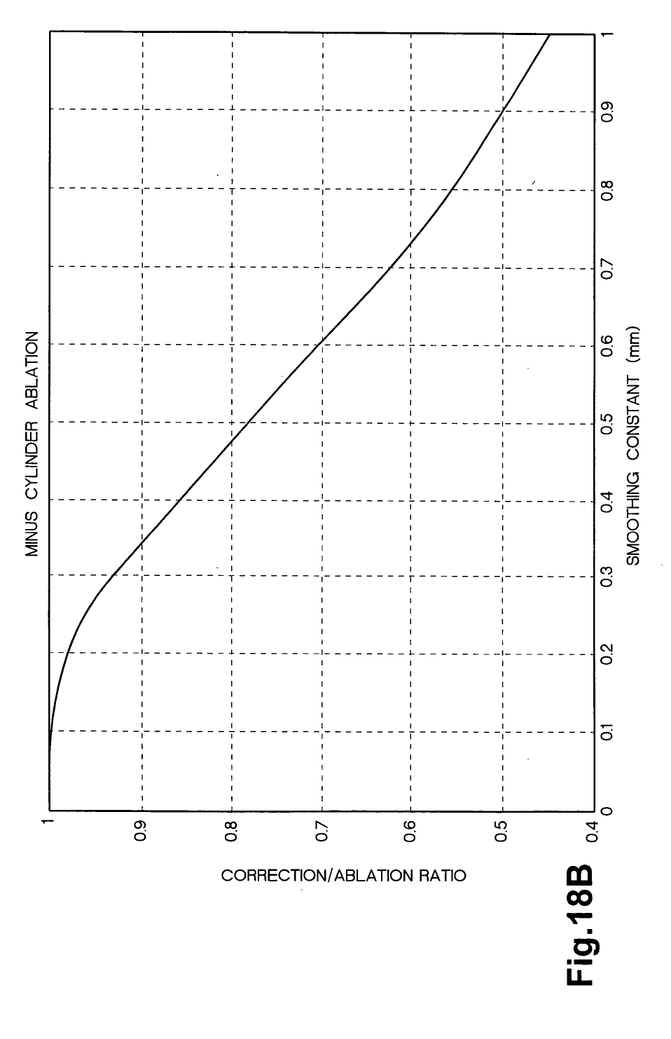

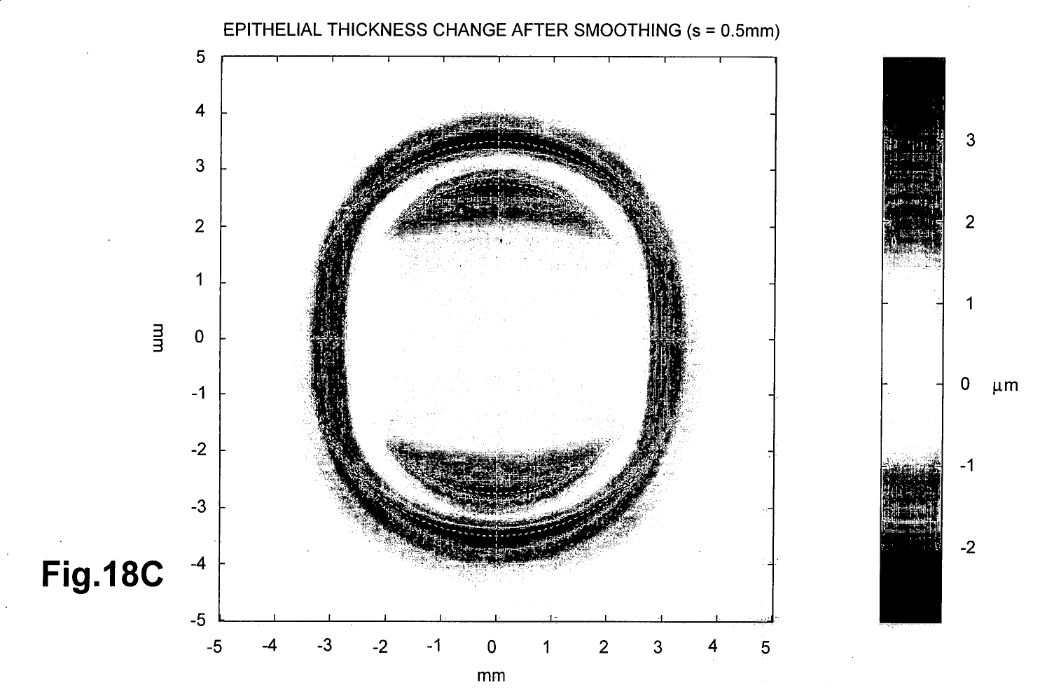

The epithelial smoothing model predicts that the minus cylinder ablation pattern of the Nidek EC-5000 (FIG. 18A) achieves less correction with increasing smoothing action (FIG. 18B). The clinical value of 0.74 for the correction/ablation ratio of 0.74 is matched at s=0.55 mm. The smoothing model also predicts an increased spherical equivalent (SE) change to 0.59 D at s=0.55 mm. The simulated epithelial thickness change is shown on FIG. 18C. The deviation from intended correction is explained by marked epithelial thickening on the flat meridian at the outer edge of the OZ. There is also epithelial thickening in the center and thinning at the outside edge of the TZ.

A believed understanding is that during corneal surface smoothing, epithelium thins over bumps or islands and thickens to fill divots or relative depressions. The mathematical model clarifies and more precisely defines this belief. The model differential equation (Equation 14) states that epithelium thins over surfaces that are more convex and thickens over surfaces that are less convex. Convexity is quantified by the local Laplacian of surface height −∇²h′. The local Laplacian is the sum of the curvature along two orthogonal tangential axes. It is also equal to twice the inverse of the radius of curvature of the best-fit sphere to the local surface. Fourier analysis shows that the differential equation is equivalent to a low-pass filter. The low-pass filter removes high-frequency (small-scale) undulations on the surface. The mathematical models agree with the believed understanding of epithelial smoothing.

The model, in accordance with the present invention, postulates that epithelium migrates towards lower areas on the cornea, with height being defined locally along the line perpendicular to the surface. It is hypothesized that epithelial cell migration is inhibited by contact with neighboring cells, and net movement toward the lower side occurs because there is less contact on that side. Contact inhibition is a well known phenomenon and has biochemical basis in cell surface molecules.

Although the model is derived from postulated epithelial behavior, it can also describe the smoothing action arising from the surface tension of the tear film. In LASIK, the “draping” effect of the flap is a potential smoothing mechanism. In PRK, subepithelial deposition of extracellular matrix material is another mechanisms for surface smoothing. In all of these mechanisms, less convex areas of the cornea are filled in.

The model explains many of the observed phenomenon after LASIK and PRK. For example, the model, in accordance with the present invention, predicts that, after myopic ablation, epithelium thickens in the center. This has been observed after myopic PRK and LASIK. The model, in accordance with the present invention, also predicts the epithelial hyperoplasia over the peripheral ablation area that occurs after hyperopic LASIK.

The model, in accordance with the present invention, also explains regression or loss of treatment effect that has been clinically observe and linked to epithelial changes. The smoothing model correctly predicts more regression should occur after hyperopic treatment than myopic treatment.

The minus cylinder ablation pattern on the Nidek EC-5000 (FIG. 18A) produces more spherical shift and less astigmatism correction than expected. This is correctly predicted by the epithelial smoothing model. A quantitative match with the correction/ablation ratio is achieved at s=0.55 mm. This agrees well with the 0.63 mm value for s estimated from the hyperopic simulations.

The model, in accordance with the present invention, also correctly predicts that oblate spherical aberration (SA) would be induced by myopic ablation.

An average value for s of 0.5 mm was determined. By averaging the estimates based on myopic, hyperopic, and cylinder ablations, the effects of extraneous variations may be reduced. The s parameter fully characterizes the corneal surface smoothing model. The ablation map a(x,y) that pre-compensates for expected surface smoothing can be calculated using a deconvolution operation.
A′=ΔH′/F′ Equation 53

“Deconvolution” is the reverse of the “convolution” operation that describes our smoothing model. Surface smoothing is described by a multiplication (Equation 48A) of the ablation map with a low-pass filter F′ in the frequency domain. Multiplication in the frequency domain is equivalent to a convolution operation in the spatial domain. Equation 53 describes a de-filtering operation in the frequency domain that removes the effect of smoothing filter F′ to achieve the desired surface change ΔH′. Direct solution of Equation 53 in the frequency domain is often unstable because F′ become zero at higher frequencies (division by zero is undefined and division by a small number amplifies noise). Thus, Equation 53 is generally solved using iterative deconvolution procedures.

The epithelial smoothing model, in accordance with the present invention, suggests that the target change map Δh(x,y) should contain a transition zone (TZ) around the OZ where there is a gradual change in curvature and slope. An abrupt change in slope would lead to an undesirable spike in radial curvature and the convexity term ∇²Δh′(x,y). Even a sharp change in curvature would lead to a step transition in the ablation. According to the model (Equation 48), it is important to design the transition zone so that the ablation does not contain much spatial frequency above 1/s (radians/mm). The hyperopia treatment would require a wider transition zone because there will be more phases of transition as well as greater slope change compared to myopia correction.

The ablation process, in accordance with the present invention, compensates for the effects of epithelial smoothing and thereby improve the accuracy of refractive correction and reduce undesirable secondary aberration. A healing-adjusted ablation design minimizes induced aberrations in laser refractive surgery. Ablation designs in accordance with the present invention can be used to correct the full range of refractive errors such as myopia (nearsightedness), hyperopia (farsightedness), astigmatism, and higher order aberrations. The present invention makes wavefront-guided treatment of aberrations more effective by anticipating and correcting secondary aberrations. It enables accurate results at the primary treatment, which is much preferred by patients over the necessity of undergoing a second surgery.

1. An apparatus for correcting optical aberrations of a patient's eye, said apparatus comprising:
a laser for performing ablation to modify the optical aberrations of the patient's eye; and
control means for controlling ablation by said laser in accordance with a control algorithm that corrects the optical aberrations and further corrects for predicted induced aberrations.

2. The apparatus of claim 1 wherein said control means includes means for storing a mathematical model that predicts the induced aberrations resulting from surface smoothing, said control means controlling said laser ablation to correct existing refractive error and aberration plus induced aberration predicted by the mathematical model.

3. The apparatus of claim 2 wherein said mathematical model is determined using a convolution operation in a spatial domain on an ablation map with a smoothing function, said mathematical model predicting the surface smoothing affect.

4. The apparatus of claim 3 wherein the smoothing function is the impulse function of a corneal surface of the patient's eye.

5. The apparatus of claim 4 wherein the mathematical model determines a corneal surface height change Δh(x′,y′) in the spatial domain according to
Δh′(x′,y′)=a′(x′,y′)\mathring{×}f′(x′,y′)
where
Δh′(x′,y′) is the corneal surface height change in local coordinates,
a′(x′,y′) is the ablation map in local coordinates, and
f′(x′,y′) is the impulse response function of the corneal surface.

6. The apparatus of claim 2 wherein said mathematical model is determined using a multiplication operation in a frequency domain on an ablation map with a frequency response of the corneal surface having a smoothing function constant that characterizes the surface smoothing, said mathematical model predicting the surface smoothing affect.

7. The apparatus of claim 6 wherein the mathematical model determines a corneal surface height change ΔH in a frequency domain according to
ΔH′(ωₓ′,ω_{y}′)=A′(ωₓ′,ω_{y}′)F′(ωₓ′, 107 _{y}′)
where
ΔH′(ωₓ′, ω_{y}′) is the Fourier transform of a corneal surface height change Δh′(x′,y′) in local coordinates,
ωₓ′ and ω_{y}′ are the respective spatial frequencies for x′ and y′ in radians/length,
A′ (ωₓ′, ω_{y}′) is the Fourier transform of a′ (x′,y′),
F′ (ωₓ′, ω_{y}′) is the frequency response of the corneal surface.

8. The apparatus of claim 7 wherein the frequency response of the corneal surface is determined by
F′(ωₓ′,ω_{y}′)=1/[1+s²(ωₓ′²+ω_{y}′²)]
where
s is a smoothing constant that characterizes the epithelial smoothing model.

9. The apparatus of claim 2 wherein said controller includes means for performing an iterative deconvolution algorithm to generate an ablation pattern that compensates for the induced aberrations.

10. The apparatus of claim 9 wherein said control algorithm provides a transition zone around an optical zone in accordance with a cubic spline function for providing continuity in ablation depth and slope.

11. The apparatus of claim 1 wherein said control means includes means for storing a rain-drop algorithm to control pulse placement of said laser during ablation.

12. A method for modifying optical refraction of a patient's eye comprising the steps of:
providing a vision correction laser to modify refraction of the patient's eye; and
controlling ablation by said laser in accordance with a control algorithm that corrects for existing measured aberrations and anticipates and corrects for induced aberrations.

13. The method of claim 12 wherein said step of controlling ablation includes determining a correction map in response to the existing measurements, storing a mathematical model that predicts corneal surface smoothing responsive to laser ablation, and determining an ablation map in response to the mathematical model.

14. The method of claim 13 wherein said step of determining the ablation map further includes generating ablation patterns using an iterative deconvolution algorithm that adjusts for a predicted smoothing response to said ablation.

15. The method of claim 12 wherein said step of controlling further includes the step using a rain-drop algorithm to control pulse placement of said laser to control ablation.

16. A method for correcting optical aberrations comprising the steps of:
providing a computer controlled ablation device for ablating a patient's eye;
performing pre-operative optical measurements to establish a corrective prescription;
establishing a mathematical model to predict surface smoothing;
establishing an ablation map based on the corrective prescription and the mathematical model; and
ablating the patient's eye with the ablation device in accordance with the ablation map.

17. The method of claim 16 wherein the step of establishing the ablation map includes establishing a transition zone based on a cubic spline function.

18. The method of claim 16 wherein the step of establishing a mathematical model includes convolving an initial ablation map with a surface smoothing function.

19. The method of claim 18 wherein the step of establishing the ablation map further includes performing an iterative deconvolution on the mathematical model.

20. The method of claim 16 further including the step of establishing a pulse sequence for the ablation device based on a rain drop algorithm.

21. A computer program product operative in a laser surgical device for correcting optical aberrations, the computer program product comprising:
a correction mapping stage that determines a correction map based on optical measurements;
an ablation mapping stage that uses a mathematical model to predict induced aberrations and adjusts the correction map for predicted induced aberrations from the mathematical model; and
a pulse control stage to optimize placement of pulses in accordance with the ablation map.

22. The computer program product of claim 21 wherein said ablation mapping stage stores the mathematical model that predicts the induced aberrations resulting from surface smoothing and wherein said pulse control stag is operative to control laser ablation to correct existing refractive error and aberration plus induced aberration predicted by the mathematical model.

23. The computer program product of claim 22 wherein said mathematical model used in the ablation mapping stage is determined using a convolution operation in a spatial domain on the ablation map with a smoothing function, said mathematical model being operative to predict the surface smoothing affect.

24. The computer program product of claim 23 wherein the smoothing function is the impulse function of a corneal surface of the patient's eye.

25. The computer program product of claim 24 wherein the mathematical model is operative to determine a corneal surface height change Δh(x′,y′) in the spatial domain according to
Δh′(x′,y′)=a′(x′,y′)\mathring{×}f′(x′,y′)
where
Δh′ (x′,y′) is the corneal surface height change in local coordinates,
a′ (x′,y′) is the ablation map in local coordinates, and
f′ (x′,y′) is the impulse response function of the corneal surface.

26. The computer program product of claim 22 wherein said mathematical model is determined using a multiplication operation in a frequency domain on an ablation map with a frequency response of the corneal surface having a smoothing function constant that characterizes the surface smoothing, said mathematical model being operative to predict the surface smoothing affect.

27. The computer program product of claim 26 wherein the mathematical model is operative to determine a corneal surface height change AH in a frequency domain according to
ΔH′(ωₓ′,ω_{y}′)=A′(ωₓ′,ω_{y}′)F′(ωₓ′,ω_{y}′)
where
ΔH′(ωₓ′, ω_{y}′) is the Fourier transform of a corneal surface height change Δh′ (x′,y′) in local coordinates,
ωₓ′ and ω_{y}′ are the respective spatial frequencies for x′ and y′ in radians/length,
A′(ωₓ′, ω_{y}′) is the Fourier transform of a′ (x′,y′),
F′ (ωₓ′, ω_{y}′) is the frequency response of the corneal surface.

28. The computer program product of claim 27 wherein the frequency response of the corneal surface is determined by
F′(ωₓ′, ω_{y}′)=1/[1+s²(ωₓ′²+ω_{y}′²)]
where
s is a smoothing constant that characterizes the epithelial smoothing model.

29. The computer program product of claim 22 wherein said ablation mapping stage is operative to perform an iterative deconvolution algorithm to generate an ablation pattern that compensates for the induced aberrations.

30. The computer program product of claim 29 wherein said ablation mapping stage provides a transition zone around an optical zone in accordance with a cubic spline function for providing continuity in ablation depth and slope.

31. The computer program product of claim 21 wherein said pulse control stage uses a rain-drop algorithm to control pulse placement during ablation.
